(19) United States
(12) Patent Application Publication
(10) Pub. No.: US 2021/0155687 A1
(43) Pub. Date: May 27, 2021

(54) DOSAGE REGIMEN

(71) Applicant: GlaxoSmithKline Intellectual Property Development Limited, Brentford, Middlesex (GB)

(72) Inventors: Juliet REID, Stevenage (GB); George SADDIC, King of Prussia, PA (US); Stefano ZAMUNER, Stevenage (GB); Chiara ZECCHIN, Stevenage (GB)

(21) Appl. No.: 16/327,751

(22) PCT Filed: Aug. 29, 2017
(86) PCT No.: PCT/EP2017/071648

§ 371 Date: Feb. 22, 2019

(30) Foreign Application Priority Data
Aug. 30, 2016 (GB) 1614627.6

(51) Int. Cl. C07K 16/24; A61K 39/395; A61K 47/12; A61K 47/18; A61K 47/02; A61K 47/26; A61K 9/08; A61P 37/06

(52) U.S. Cl. CPC C07K 16/248; A61K 39/3955; A61K 47/12; A61K 47/183; A61K 47/02; A61K 2039/505; A61K 9/08; A61P 37/06; C07K 2317/76; C07K 2317/51; C07K 2317/515; A61K 47/26

(57) ABSTRACT

The present invention provides pharmaceutical compositions comprising antigen binding proteins that specifically bind Oncostatin M (OSM) and in particular human OSM (hOSM) and which inhibit the binding of OSM to the gp130 receptor, novel therapeutic regimens for said pharmaceutical compositions; and methods for administering said pharmaceutical compositions in the treatment of an inflammatory or autoimmune disorder, in particular in the treatment of systemic sclerosis.

### FIELD OF THE INVENTION

The present invention relates to pharmaceutical compositions comprising antigen binding proteins that specifically bind Oncostatin M (OSM) and in particular human OSM (hOSM); novel therapeutic regimens for said pharmaceutical compositions; and methods for administering said pharmaceutical compositions in the treatment of an inflammatory or autoimmune disorder, in particular in the treatment of systemic sclerosis.

### BACKGROUND OF THE INVENTION

Oncostatin M is a 28 kDa glycoprotein that belongs to the interleukin 6 (IL-6) family of cytokines, which includes IL-6, Leukaemia Inhibitory Factor (LIF), ciliary neurotrophic factor (CNTF), cardiotropin-1 (CT-1) and cardiotrophin-1 like cytokine (See Kishimoto T et al. (1995) Blood 86: 1243-1254), which share the gp130 transmembrane signalling receptor (See Taga T and Kishimoto T (1997) Annu. Rev. Immunol. 15: 797-819). OSM was originally discovered by its ability to inhibit the growth of the melanoma cell line A375 (See Malik N (1989) et al. Mol Cell Biol 9: 2847-2853). Subsequently, more effects were discovered and it was found to be a multifunctional mediator, like other members of the IL-6 family. OSM is produced in a variety of cell types including macrophages, activated T cells (See Zarling J M (1986) PNAS (USA) 83: 9739-9743), polymorphonuclear neutrophils (See Grenier A et al. (1999) Blood 93:1413-1421), eosinophils (See Tamura S et al. (2002) Dev. Dyn. 225: 327-31), and dendritic cells (See Suda T et al. (2002) Cytokine 17:335-340). It is also expressed in the pancreas, kidney, testes, spleen, stomach, brain (See Znoyko I et al. (2005) Anat Rec A Discov Mol Cell Evol Biol 283: 182-186), and bone marrow (See Psenak O et al. (2003) Acta Haematol 109: 68-75). Its principle biological effects include activation of endothelium (See Brown T J et al. (1993) Blood 82: 33-7), activation of the acute phase response (See Benigni F et al. (1996) Blood 87: 1851-1854), induction of cellular proliferation or differentiation, modulation of inflammatory mediator release, haematopoesis (See Tanaka M et al. (2003) 102: 3154-3162), re-modelling of bone (See de Hooge A S K (2002) Am J Pathol 160: 1733-1743), promotion of angiogenesis (See Vasse M et al. (1999) Arterioscler Thromb Vasc Biol 19:1835-1842) and wound healing.

Receptors for OSM (OSM receptor, OSM receptor β, “OSMRβ”) are expressed on a wide range of cells including epithelial cells, chondrocytes, fibroblasts (See Langdon C et al. (2003) J Immunol 170: 548-555), and cells from neuronal smooth muscle, lymph node, bone, heart, small intestine, lung and kidney (See Tamura S et al. (2002) Mech Dev 115: 127-131). Several lines of evidence suggest that endothelial cells are a primary target for OSM. These cells express 10 to 20 fold higher numbers of both high and low affinity receptors for OSM and exhibit profound and prolonged alterations in phenotype following stimulation with OSM (See Modur V et al. (1997) J Clin Invest 100: 158-168). In addition, OSM is a major autocrine growth factor for Kaposi's sarcoma cells, which are thought to be of endothelial origin (See Murakami-Mori K et al. (1995) J Clin Invest 96:1319-1327).

In common with other IL-6 family cytokines, OSM binds to the transmembrane signal transducing glycoprotein, gp130. A key feature of the gp130 cytokines is the formation of oligomeric receptor complexes that comprise gp130 and one or more co-receptors depending on the ligand (Reviewed in Heinrich P C et al. (2003) Biochem J. 374: 1-20). As a result, these cytokines can mediate both the shared and unique biological activities in vitro and in vivo depending on the composition of the receptor complex formed. Human OSM (hOSM) differs from the other IL-6 cytokines in that it can form complexes with gp130 and either one of the two co-receptors, LIFR or the oncostatin receptor (OSMR).

The crystal structure of hOSM has been solved and shown to comprise a four a helical bundle with two potential glycosylation sites. Two separate ligand binding sites have been identified by site-directed mutagenesis on the hOSM molecule (See Deller M C et al. (2000) Structural Fold Des. 8:863-874). The first, called Site II (sometimes “site 2”) interacts with gp130 and the second site, called Site III (sometimes “site 3”), at the opposite end of the OSM molecule, interacts with either LIFR or OSMR. Mutagenesis experiments have shown that the binding sites for LIFR and OSMR are almost identical but that a single amino acid mutation can discriminate between the two.

There is increasing evidence to support the hypothesis that modulating OSM-gp130 interaction may be of benefit in the treatment of inflammatory diseases and disorders, such as systemic sclerosis, rheumatoid arthritis, osteoarthritis, idiopathic pulmonary fibrosis, pain, inflammatory lung disease, cardiovascular disease and psoriasis.

It is therefore an object of the present invention to provide a therapeutic approach to the treatment of for example systemic sclerosis, ulcerative colitis, inflammatory bowel disease or rheumatoid arthritis. Also treatment of chronic inflammatory diseases and disorders such as osteoarthritis, idiopathic pulmonary fibrosis, cancer, asthma, pain, cardiovascular disease and psoriasis. In particular, it is an object of the present invention to provide antigen binding proteins that specifically bind OSM (e.g. hOSM, particularly Site II thereof) and modulate (i.e. inhibit or block) the interaction between OSM and gp130 in the treatment of diseases and disorders responsive to modulation of that interaction.

Systemic sclerosis (SSc) is a multisystem autoimmune disease, in which the interrelated processes of inflammation, fibrosis and microvascular damage result in a complex pattern of organ-based complications with high mortality and morbidity. Symptoms include hardening, scarring and blistering. There are no approved drugs for the treatment of SSc and, as such, it remains an area of great unmet medical need [Denton, 2013].

### SUMMARY OF THE INVENTION

The present invention provides novel dosing regimens for treating an inflammatory or autoimmune disorder or disease, such as systemic sclerosis or rheumatoid arthritis with an anti-OSM antibody.

The present invention discloses a pharmaceutical composition comprising an antigen binding protein which is capable of binding to OSM and inhibits the binding of OSM to the gp130 receptor, and wherein an effective dose of said pharmaceutical composition comprises 50-300 mg of said antigen binding protein.

The present disclosure also encompasses methods of treating a human patient afflicted with an inflammatory or autoimmune disease by administering said pharmaceutical composition to said patient.

The present invention further provides methods of administering pharmaceutical compositions comprising antigen binding proteins which are capable of binding to OSM, for example which specifically bind to human OSM (hOSM) and which inhibit the binding of OSM to the gp130 receptor to a human.

The present invention provides pharmaceutical compositions comprising antigen binding proteins which are capable of binding to OSM, for example which specifically bind to human OSM (hOSM) and which inhibit the binding of OSM to the gp130 receptor and wherein an effective dose of said pharmaceutical compositions comprises 50-300 mg of said antigen binding proteins.

### DESCRIPTION OF FIGURES

FIG. 1 shows observed versus predicted mean total OSM at different dose levels.

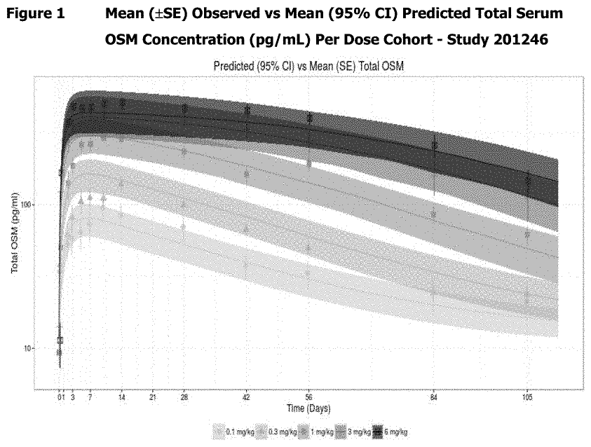

FIG. 2 is a TMDD model to derive mean (95% CI) target engagement (% TE).

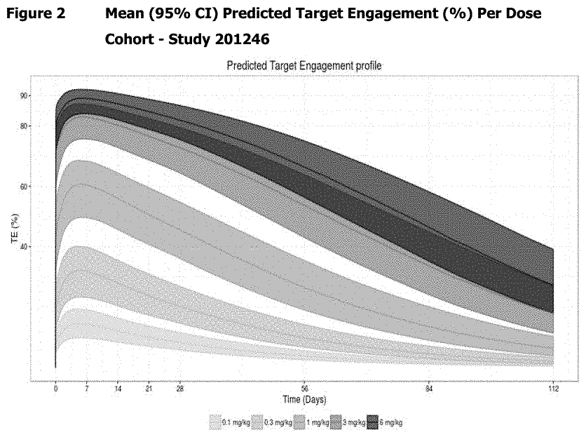

FIG. 3 illustrates the simulated target engagement profile of mAb 1 during repeat dosing based on the one compartment PK-TE model.

FIGS. 4 & 5 show a single more concentrated administration as per Table 4 in both plasma and blister fluid.

FIGS. 6 & 7 shows target engagement in both Plasma and blister fluid.

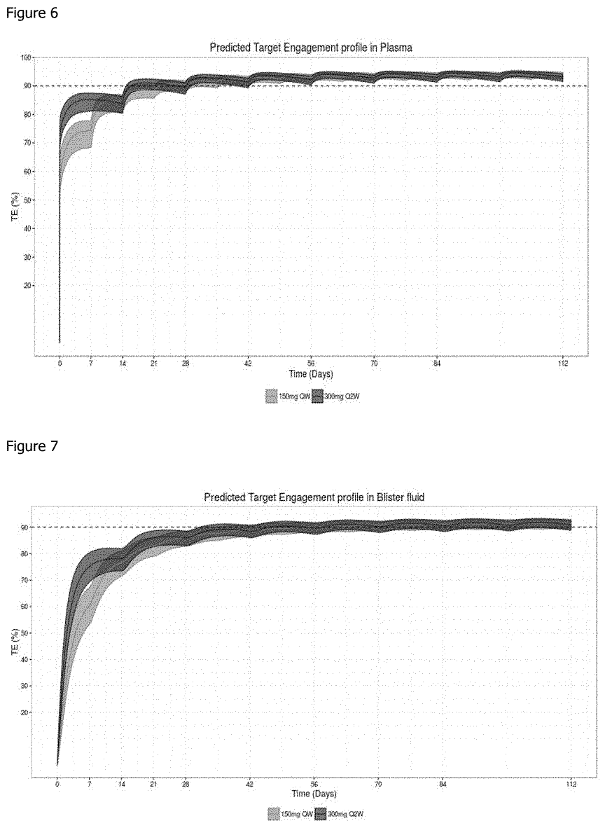

FIG. 8 shows percentage and mean changes from baseline of a dose related decrease in platelet number.

FIG. 9 shows that a 3 mg/kg (SC) dosage in said FTIH mab1 study produced a 35% reduction and a 6 mg/kg (SC) dosage a 60% reduction.

### DETAILED DESCRIPTION OF THE INVENTION

The present invention provides a pharmaceutical composition comprising an antigen binding protein which is capable of binding to OSM, for example which specifically binds to human OSM (hOSM), and which inhibits the binding of OSM to the gp130 receptor and wherein an effective dose of said pharmaceutical composition comprises 50-300 mg of said antigen binding protein. For example, in one embodiment the effective dose of said pharmaceutical composition comprises 100 mg or 150 mg or 200 mg or 300 mg of said antigen binding protein.

The present invention also provides a pharmaceutical composition for use in the treatment of inflammatory or autoimmune disorders or diseases, such as systemic sclerosis, wherein the pharmaceutical composition comprises from about 50 mg to about 300 mg of an antigen binding protein which is capable of binding to OSM, for example which specifically binds to hOSM and which inhibits the binding of OSM to the gp130 receptor, wherein the pharmaceutical composition is for administration once a week or once every other week. For example, in one embodiment the pharmaceutical composition comprises 100 mg or 150 mg or 200 mg or 300 mg of said antigen binding protein.

In one embodiment, the invention provides a method for treating an autoimmune or inflammatory disease (e.g. systemic sclerosis, ulcerative colitis or inflammatory bowel disease) in a patient, comprising administering to the patient an OSM binding protein (e.g. an anti-OSM antibody) which specifically binds to hOSM and which inhibits the binding of OSM to the gp130 receptor, at a dose of 50-300 mg of said antigen binding protein. For example, in one embodiment the effective dose of said pharmaceutical composition comprises 50 mg, or 100 mg or 150 mg or 200 mg or 300 mg of said antigen binding protein. For example, in one embodiment the effective dose of said pharmaceutical composition comprises 100 mg of said antigen binding protein. For example, in one embodiment the effective dose of said pharmaceutical composition comprises 150 mg of said antigen binding protein.

In another embodiment, the pharmaceutical composition of the present invention can be administered to a human daily, every other day, weekly, every other week, every 4 weeks, or once a month. In a further embodiment, the pharmaceutical composition of the present invention can be administered to a human weekly.

In a further embodiment, the pharmaceutical composition of the present invention can be administered to a human every other week.

In another embodiment, the pharmaceutical composition of the present invention can be administered to a human once daily, once every other day, once every seven days, once every fourteen days, once every 4 weeks, or once every month.

In a further embodiment, the pharmaceutical composition is administered once every 7 days.

In a further embodiment, the pharmaceutical composition is administered once every 14 days.

In another embodiment, methods are provided for administering at least one antigen binding protein which specifically binds to hOSM to a human comprising administering a pharmaceutical composition of the invention to the human. In one embodiment, the pharmaceutical composition is administered subcutaneously. The pharmaceutical composition can be administered as a subcutaneous injection of at least 1.0 mL injection solution. In one embodiment, the antigen binding protein is administered in two or three injections which may be the same dose or different doses of the same pharmaceutical composition. The pharmaceutical composition may be administered at the same or different injection sites. Subcutaneous injections of the present invention may be administered as single injections wherein the entire dose is administered as a single shot, wherein the entire volume of the shot is administered all at once. A single shot injection may be administered multiple times. A single shot differs from a continuous or titrated administration, e.g. an infusion, wherein the administration may be administered over several minutes, hours or days.

In one embodiment, the pharmaceutical composition is administered as a monotherapy. In another embodiment, the pharmaceutical composition is co-administered with standard of care medicaments such as, for example, corticosteroids, prednisone, or methotrexate.

As is understood in the art, various methods can be employed to collect, measure and assess pharmacokinetic and pharmacodynamic data in the blood, plasma and/or other tissue. In one embodiment, the measurements are taken from blister fluid. Mechanistic biomarkers of fibrosis, inflammation and vasculopathy may be measured in blood and/or skin in order to provide evidence of the modulation of key biological pathways involved in the pathogenesis of systemic sclerosis.

In an aspect of the present invention there is provided a method of treating a human patient afflicted with an inflammatory disease or disorder which method comprises the step of administering to said patient a therapeutically effective amount of the pharmaceutical composition of the invention. In a further embodiment, the pharmaceutical composition as described herein is for the treatment of systemic sclerosis. In a further embodiment, the pharmaceutical composition as described herein is for the treatment of ulcerative colitis or inflammatory bowel disease.

In one embodiment, there is provided use of an antigen binding protein as described herein in the manufacture of a medicament for the treatment of an inflammatory or autoimmune disorder or disease. In a further embodiment, the pharmaceutical composition as described herein is for the treatment of Systemic sclerosis. In a further embodiment, the pharmaceutical composition as described herein is for the treatment of ulcerative colitis or inflammatory bowel disease.

In another embodiment, there is provided a pharmaceutical composition for use in the treatment of an inflammatory or autoimmune disorder or disease.

The dose and duration of treatment relates to the relative duration of the antigen binding proteins of the present invention in the human circulation, the condition being treated and the general health of the patient. It is envisaged that repeated dosing over an extended time period (e.g. two to six months) may be required to achieve maximal therapeutic efficacy. In one embodiment, the pharmaceutical composition is administered chronically.

The optimal dose and administration of the antigen binding protein, in particular an anti-OSM antibody, will depend on the characteristics and properties of the antigen binding protein. The affinity of an antibody is often important to determine whether a target will be successfully blocked or neutralised. However, despite an antibody binding and blocking a target in vitro, it is often the case that the antibody fails in the clinical to meet the necessary endpoints in vivo. The exemplified dosage regimen and administration protocol as herein described has been determined using analysis of blister fluid rather than simply plasma analysis leading to a potentially more accurate indication of the levels of OSM and antigen binding protein in the relevant compartments of the body and therefore its effect on managing disease.

In another aspect of the present invention there is provided a kit-of-parts comprising the antigen binding protein or pharmaceutical composition according to the invention described herein together with instructions for use.

In an embodiment, the disease or disorder is selected from the group consisting of systemic sclerosis, ulcerative colitis or inflammatory bowel disease, rheumatoid arthritis, osteoarthritis, Psoriasis, Idiopathic Pulmonary Fibrosis or Multiple Sclerosis. In an embodiment, the disease or disorder is selected from the group consisting of systemic sclerosis, ulcerative colitis or inflammatory bowel disease.

In yet a further embodiment the pharmaceutical composition comprises sodium acetate, EDTA, arginine, sodium chloride and polysorbate 80 (PS80) and has a pH of 5.5. In yet a further embodiment the pharmaceutical composition comprises the antigen binding protein at a concentration of 100 mg/ml, 50 mM sodium acetate, 0.05 mM EDTA, 1.0% arginine, 51 mM sodium chloride, and 0.02% PS80 at a pH 5.5 In yet a further embodiment the pharmaceutical composition comprises the antigen binding protein at a concentration of 150 mg/ml, 50 mM sodium acetate, 0.05 mM EDTA, 1.0% arginine, 51 mM sodium chloride, and 0.02% polysorbate 80 at a pH of 5.5.

The invention further provides a pharmaceutical composition comprising an antigen-binding protein as described herein and a pharmaceutically acceptable carrier.

The antigen binding proteins described herein can be lyophilized for storage and reconstituted in a suitable carrier prior to use. This technique has been shown to be effective with conventional antigen binding proteins and art-known lyophilization and reconstitution techniques can be employed.

The antigen binding proteins of the present invention are related to, or derived from a murine monoclonal antibody (mab), 10G8. The 10G8 murine heavy chain variable region is encoded by SEQ ID NO: 7 and the 10G8 murine light chain variable region is encoded by SEQ ID NO: 9.

The 10G8 murine heavy chain variable region is provided by SEQ ID NO: 8 and the 10G8 murine light chain variable region is provided by SEQ ID NO: 10.

In one embodiment of the present invention the antigen binding protein is a human, humanised or chimeric antibody. In a further embodiment, the antibody is humanised. In one embodiment, the antibody is a monoclonal antibody.

The heavy chain variable regions (VH) of the antigen binding protein may comprise the following CDRs or variants of these CDRs as defined by Kabat (Kabat et al.; Sequences of proteins of Immunological Interest NIH, 1987)):

CDRH1 of SEQ ID NO: 1 or SEQ ID NO:43

CDRH2 of SEQ ID NO: 2

CDRH3 of SEQ ID NO: 3

The light chain variable regions (VL) of the present invention may comprise the following CDRs or variants of these CDRs as defined by Kabat (Kabat et al.; Sequences of proteins of Immunological Interest NIH, 1987)):

CDRL1 of SEQ ID NO: 4

CDRL2 of SEQ ID NO: 5 or SEQ ID NO:44

CDRL3 of SEQ ID NO: 6

In a further embodiment of the invention the antigen binding protein comprises CDRH3 of SEQ. ID. NO: 3, CDRH2 of SEQ. ID. NO: 2, CDRL1 of SEQ. ID. NO: 4 and CDRL3 of SEQ. ID. NO: 6 and may further comprise CDRH1 of SEQ. ID. NO: 1 or SEQ ID NO:43 and CDRL2 of SEQ. ID. NO: 5 or SEQ ID NO: 44.

In another embodiment, the antigen binding protein comprises CDRH3 of SEQ. ID. NO: 3, CDRH2 of SEQ. ID. NO: 2, CDRL1 of SEQ. ID. NO: 4, CDRL2 of SEQ. ID. NO: 5 and CDRL3 of SEQ. ID. NO: 6.

In yet another embodiment the antigen binding protein comprises CDRH3 of SEQ. ID. NO: 3, CDRH2 of SEQ. ID. NO: 2, CDRH1 of SEQ. ID. NO: 1, CDRL1 of SEQ. ID. NO: 4, CDRL2 of SEQ. ID. NO: 5 and CDRL3 of SEQ. ID. NO: 6.

In yet another embodiment the antigen binding protein comprises CDRH3 of SEQ. ID. NO: 3, CDRH2 of SEQ. ID. NO: 2, CDRH1 of SEQ. ID. NO: 1, CDRL1 of SEQ. ID. NO: 4, CDRL2 of SEQ. ID. NO: 44 and CDRL3: SEQ. ID. NO: 6.

In yet another embodiment the antigen binding protein comprises CDRH3 of SEQ. ID. NO: 3, CDRH2 of SEQ. ID. NO: 2, CDRH1 of SEQ. ID. NO: 43, CDRL1 of SEQ. ID. NO: 4, CDRL2 of SEQ. ID. NO: 5 and CDRL3: SEQ. ID. NO: 6.

In yet another embodiment the antigen binding protein comprises CDRH3 of SEQ. ID. NO: 3, CDRH2 of SEQ. ID. NO: 2, CDRH1 of SEQ. ID. NO: 43, CDRL1 of SEQ. ID. NO: 4, CDRL2 of SEQ. ID. NO: 44 and CDRL3 of SEQ. ID. NO: 6.

In one embodiment, the antigen binding protein does not interact directly via CDRH1 or CDRL2 with OSM.

In a further embodiment of the invention there is provided an antigen binding protein comprising an isolated heavy chain variable domain of SEQ ID NO:19 and an isolated light chain variable domain of SEQ ID NO:27.

In one embodiment, the antigen binding protein of the present invention comprises a heavy chain variable region encoded by SEQ. ID. NO:20 and a light chain variable region encoded by SEQ. ID. NO:28.

In one embodiment, the antigen binding protein of the present invention comprises a heavy chain encoded by SEQ. ID. NO:41 and a light chain variable region encoded by SEQ. ID. NO:37.

In one embodiment, the antigen binding protein of the present invention comprises a heavy chain of SEQ. ID. NO:42 and a light chain variable region of SEQ. ID. NO:38.

### Definitions

The terms “antigen binding protein” and “OSM binding protein” are used interchangeably and as used herein refer to antibodies, antibody fragments for example a domain antibody (dAb), ScFv, FAb, FAb₂, and other protein constructs. For example the antigen binding protein or OSM binding protein is capable of binding to OSM. For example specifically the antigen binding protein or OSM binding protein binds to OSM and inhibits the binding of OSM to the gp130 receptor. Antigen binding molecules may comprise at least one Ig variable domain, for example antibodies, domain antibodies (dAbs), Fab, Fab′, F(ab′)₂, Fv, ScFv, diabodies, mAbdAbs, affibodies, heteroconjugate antibodies or bispecific antibodies. In one embodiment, the antigen binding molecule is an antibody. In another embodiment, the antigen binding molecule is a dAb, i.e. an immunoglobulin single variable domain such as a VH, VHH or VL that specifically binds an antigen or epitope independently of a different V region or domain. Antigen binding molecules may be capable of binding to two targets, i.e. they may be dual targeting proteins. Antigen binding molecules may be a combination of antibodies and antigen binding fragments such as, for example, one or more domain antibodies and/or one or more ScFvs linked to a monoclonal antibody. Antigen binding molecules may also comprise a non-Ig domain for example a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to OSM. As used herein an “antigen binding protein” will be capable of antagonising and/or neutralising human OSM. In addition, an antigen binding protein may inhibit and/or block OSM activity by binding to OSM and preventing it from binding and/or activating the gp130 receptor.

The terms Fv, Fc, Fd, Fab, or F(ab)₂ are used with their standard meanings (see, e.g., Harlow et al., Antibodies A Laboratory Manual, Cold Spring Harbor Laboratory, (1988)).

The term “antibody” is used herein in the broadest sense and specifically covers monoclonal antibodies (including full length monoclonal antibodies), polyclonal antibodies, and multispecific antibodies (e.g. bispecific antibodies)

The term “monoclonal antibody” as used herein refers to an antibody obtained from a population of substantially homogenous antibodies i.e. the individual antibodies comprising the population are identical except for possible naturally occurring mutations that may be present in minor amounts. Monoclonal antibodies are highly specific being directed against a single antigenic binding site. Furthermore, in contrast to polyclonal antibody preparations which typically include different antibodies directed against different determinants (epitopes), each monoclonal antibody is directed against a single determinant on the antigen.

A “chimeric antibody” refers to a type of engineered antibody in which a portion of the heavy and/or light chain is identical with or homologous to corresponding sequences in antibodies derived from a particular donor antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567 and Morrison et al. Proc. Natl. Acad. Sci. USA 81:6851-6855) (1984)).

A “humanised antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

Throughout the present specification and the accompanying claims the term “comprising” and “comprises” incorporates “consisting of” and “consists of”. That is, “comprising” and “comprises” are intended to convey the possible inclusion of other elements or integers not specifically recited, where the context allows.

The term “specifically binds” as used throughout the present specification in relation to antigen binding proteins of the invention means that the antigen binding protein binds human OSM (hOSM) with no or insignificant binding to other human proteins. The term however does not exclude the fact that antigen binding proteins of the invention may also be cross-reactive with other forms of OSM, for example primate OSM.

The term “directly interacts” as used throughout this specification in relation to antigen binding proteins of the invention means that when the antigen binding protein is bound to human OSM (hOSM) that specific residues on the antigen binding protein are within 3.5 Å of specific residues on the hOSM.

The term “inhibits” as used throughout the present specification in relation to antigen binding proteins of the invention means that the biological activity of OSM is reduced in the presence of the antigen binding proteins of the present invention in comparison to the activity of OSM in the absence of such antigen binding proteins. Inhibition may be due, but not limited to, one or more of: blocking OSM and receptor binding, preventing the OSM from activating the receptor, down regulating OSM, or affecting effector functionality. The antibodies of the invention may neutralise OSM.

“CDRs” are defined as the complementarity determining region amino acid sequences of an antibody which are the hypervariable domains of immunoglobulin heavy and light chains. There are three heavy chain and three light chain CDRs in the variable portion of an immunoglobulin. Thus, “CDRs” as used herein may refer to all three heavy chain CDRs, or all three light chain CDRs (or both all heavy and all light chain CDRs, if appropriate).

CDRs provide the majority of contact residues for the binding of the antibody to the antigen or epitope. CDRs of interest in this invention are derived from donor antibody variable heavy and light chain sequences, and include analogues of the naturally occurring CDRs, e.g. analogues of murine 10G8 CDRs (SEQ ID NO:1-6), which analogues also share or retain the same antigen binding specificity and/or neutralizing ability as the donor antibody from which they were derived, e.g. 10G8.

The CDR sequences of antibodies can be determined by the Kabat numbering system (Kabat et al.; (Sequences of proteins of Immunological Interest NIH, 1987), alternatively they can be determined using the Chothia numbering system (Al-Lazikani et al., (1997) JMB 273, 927-948), the contact definition method (MacCallum R. M., and Martin A. C. R. and Thornton J. M., (1996), Journal of Molecular Biology, 262 (5), 732-745) or any other established method for numbering the residues in an antibody and determining CDRs known to the person skilled in the art.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

| | | | | | Minimum |
|---|---|---|---|---|---|
| | Kabat | Chothia | AbM | Contact | binding |
| | CDR | CDR | CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Throughout this specification, amino acid residues in antibody sequences are numbered according to the Kabat scheme. Similarly, the terms “CDR”, “CDRL1”, “CDRL2”, “CDRL3”, “CDRH1”, “CDRH2”, “CDRH3” follow the Kabat numbering system as set forth in Kabat et al.; Sequences of proteins of Immunological Interest NIH, 1987.

The terms “VH” and “VL” are used herein to refer to the heavy chain variable domain and light chain variable domain, respectively, of an antibody.

As used herein the term “domain” refers to a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. An “antibody single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (VH, VHH, VL) that specifically binds an antigen or epitope independently of a different variable region or domain.

The term “Effector Function” as used herein is meant to refer to one or more of antibody dependant cell mediated cytotoxic (ADCC) activity, complement-dependant cytotoxic (CDC) activity, Fc-mediated phagocytosis and antibody recycling via the FcRn receptor.

The present invention is now described by way of example only.

### EXAMPLES

The following examples illustrate various non-limiting aspects of the invention. For the following examples, unless noted otherwise, the antigen binding protein is an antibody having a heavy chain according to SED ID NO: 42 and light chain according to SEQ ID NO: 38 and is hereafter referred to as “mab 1”. Mab 1 in these examples was formulated as 1.2 mL fill with 1 mL extractable volume at 100 mg/mL, with 50 mM sodium acetate, 0.05 mM EDTA, 1.0% arginine, 51 mM sodium chloride and 0.02% polysorbate 80 at a pH of 5.5.

### Example 1: Mab 1 Phase II Clinical Protocol

In this prophetic example a placebo-controlled, repeat-dose, proof of mechanism study to evaluate the safety, tolerability, pharmacokinetics, pharmacodynamics and to explore efficacy of mab 1 in participants with diffuse cutaneous systemic sclerosis will be carried out.

Participants with diffuse cutaneous systemic sclerosis (dcSSc), with active disease and a disease duration of ≤60 months, will be enrolled. Active disease defined by at least one of the following criteria at screening:
C reactive protein (CRP) ≥6 mg/l (0.6 mg/dL), that in the opinion of the investigator is due to SSc.
Disease duration ≤18 months at screening, defined as time from the first non-Raynaud's phenomenon manifestation.
Increase of ≥3 mRSS units, compared with an assessment performed within the previous 6 months.
Involvement of one new body area and an increase of ≥2 mRSS units compared with an assessment performed within the previous 6 months.
Involvement of two new body areas within the previous 6 months.

Participants will be dosed subcutaneously at one of two dose levels, every other week, for at least 10 weeks with either mab 1 or placebo. The duration of the study, including screening, will be approximately 32 weeks, for all participants. In total a minimum of 24 participants and a maximum of 40 participants will be enrolled across two cohorts. A participant in cohort 1 or cohort 2 is considered evaluable for study endpoints if they have received at least 4 doses of mab 1 or placebo and have had biopsies at both the Day 1 and the Day 85 (Week 12) assessment. Additional participants may be randomised into the study at the discretion of the sponsor up to a maximum of 40 participants in the study overall.

As this is the first study of mab 1 in participants with SSc, and the first repeat dose study, the primary endpoint is the safety and tolerability of mab1. In addition, this study will include assessments of the pharmacokinetics, target engagement and downstream pharmacology of mab1. This will be achieved by assessing mab1 and OSM levels in blood and skin blister fluid and mRNA markers of mab1 pharmacology in skin biopsies. Skin involvement is emphasised because it is readily studied, contributes substantially to the morbidity experienced by patients with dcSSc and exemplifies the three major pathological processes involved in the condition. Usually plasma levels of mab 1 and OSM are measured for such assessment. However, a technique to allow monitoring of levels in the blister fluid has been devised. This method allows more accurate analysis in the tissues and since mab 1 yield in blister fluid is typically 20-30% lower than would be measured in blood, a better prediction of dosage efficacy is possible.

The assessment of biomarkers of fibrosis, inflammation and vasculopathy in blood and skin biopsies will also be performed, and for this reason the population is enriched for early active disease. Changes in these parameters and their association with each other and with preliminary measures of clinical efficacy will be assessed. This data is intended to provide evidence that mab1 is having an impact on key pathways involved in the pathogenesis of SSc.

The purpose of cohort 1 is to evaluate the safety and tolerability of repeat doses of a pharmacologically active but submaximal dose of mab 1, before escalating to a higher dose.

The duration of the Treatment Phase is based on the expectation that an effective therapy should cause changes in the mechanistic parameters at this timepoint of approximately 10-12 weeks.

The mab1 half-life (t½) is between 19 and 25 days, consistent with a typical monoclonal antibody half-life for a soluble cytokine approximately 16 weeks after the last administration of mab 1.

The placebo group is required for a valid evaluation of adverse events attributable to mab 1 treatment versus those independent of mab 1 treatment. The placebo participants will also serve as negative controls for the biomarker and efficacy assessments.

Participants will be randomised in a 3:1 ratio to mab 1 and placebo respectively. This unbalanced allocation ratio means that more participants are available for the assessment of within-participant changes in biomarkers after dosing with mab 1 and allows more participants to receive mab 1.

Participants will be allowed to continue with some background therapies, including mycophenolate and low dose oral corticosteroids to avoid excluding potential participants in this rare disease. Other immunosuppressive treatments will be excluded, in order to minimise inter-participant variability in this small trial.

Dose levels for this study have been selected on the basis of PK/PD predictions, data from the first time in human study with mab 1, and preclinical data (See Example 2 for detail). Two dose levels (100 mg and 300 mg) have been selected based on predicted target engagement in both serum and skin compartments. Further planned dosage studies with 150 mg will also be used in a weekly or every other week administration regimen.

An in-vivo affinity of approximately 0.6 nM was estimated from first time in human (FTIH) data. The typical mab 1 apparent distribution volume was 11.5 l (95% CI: 10.2-13.1) and the typical apparent systemic clearance was 14.1 ml/hr (95% CI: 12.7-15.6).

TABLE 2

| | | |
|---|---|---|
| Study Treatment Name: | mab 1 | Placebo |
| Dosage formulation: | Solution for injection; 50 | Normal saline (0.9% w/v |
| | mM sodium acetate, | sodium chloride) |
| | 0.05 mM EDTA, 1.0% | |
| | arginine, 51 mM sodium | |
| | chloride, pH 5.5 with | |
| | 0.02% polysorbate 80. | |
| Unit dose | 1.2 mL fill with 1 mL | Not applicable |
| strength(s)/Dosage | extractable volume at | |
| level(s): | 100 mg/mL | |
| Route of Administration | SC injection only | SC injection only |
| Dosing instructions: | Administered by | Administered by investigator |
| | investigator or designee. | or designee. Injection |
| | Cohort 1: 100 mg dose | volume and number of |
| | 1 × 1 ml injected via | injections will match active |
| | needle and syringe. | doses administered. |
| | Cohort 2: 300 mg dose | |
| | 3 × 1 ml injected via | |
| | needle and syringe. | |
| | If a dose lower than 300 | |
| | mg is required then the | |
| | volume injected will be | |
| | reduced accordingly. | |

### Example 2: Mab 1 Dose Selection

Mab 1 dose levels (which are summarised in Table 2) were selected based on PK/PD predictions and preclinical data form the FTIH study wherein dosages of 0.1, 0.3, 0.6, 1.0, 3.0 and 6.0 mg/kg were used.

The ‘Minimal Anticipated Biological Effect Level’ (MABEL), as per Guideline on Strategies to Identify and Mitigate Risks for First-In-Human Clinical Trials with Investigational Medicinal Products [CHMP, 2007] was used to define the starting dose and is defined as the dose level predicted to result in a maximum inhibition in plasma in the 20-40% range. A previous anti-OSM antibody (mab 2) had a favourable safety and tolerability profile in both healthy volunteers and rheumatoid arthritis patients at doses that achieved up to 90% target engagement (TE).

A dose of 0.1 mg/kg for mab 1 was designated the MABEL dose as the maximum predicted PD inhibition was 41%, according to human PK/PD predictions in the best case scenario.

The highest planned dose of 6 mg/kg of mab 1 was expected to provide full TE in plasma (defined as >90%) lasting 14 to 40 days, with lower TE levels (<90%) predicted to be achieved in tissue compartments, including skin.

The cynomolgus monkey study for mab 1, used in the non-clinical assessment of pharmacology and toxicology, provided reasonable assurance that there were no undue or unforeseen risks for the first administration of mab 1 to humans, at the dose levels used in this study. The highest planned exposures (Cₘₐₓ 33.3 μg/mL, AUC(0-∞)=23534 μg*h/mL) were predicted to be almost 100-fold below the safety margin provided by the toxicology study—See Table 3.

TABLE 3
mAb 1 Doses, Predicted Exposures and Safety Margin

| | Predicted PK and PK/PD inhibition | | | | | |
|---|---|---|---|---|---|---|
| | Max OSM | Max OSM | | | | |
| Dose | Inhibition | Inhibition | Cₘₐₓ | AUC(0-∞) | Safety Margi [symbol] | |
| (mg/kg) | (%) (plasma) | (%) (skin) | (μg/mL) | (μg*h/mL) | Cₘₐₓ | AUC |
| 0.1 | 18 | 2-8 | 0.55 | 392 | 14291x | 5689 [symbol] |
| 0.3 | 40 | 5-22 | 1.7 | 1177 | 4737x | 1895 [symbol] |
| 1 | 69 | 14-49 | 5.5 | 3922 | 1417x | 569 [symbol] |
| 3 | 87 | 33-75 | 16.7 | 11767 | 472x | 190 [symbol] |
| 6 | 93 | 50-86 | 33.3 | 23534 | 236x | 95x |

[symbol]

indicates data missing or illegible when filed

Pharmacodynamics has been assessed by measurement of free and total OSM levels to characterise target engagement in the single ascending dose FTIH study. A target mediated drug disposition (TMDD) model using a one-compartment PK model together with binding kinetics of drug and target was developed to assess the in vivo affinity of mAb 1 to OSM in serum. In this study, free OSM levels were below the limit of quantitation after drug administration indicating substantial OSM inhibition. Error! Reference source not found. shows

Estimated in-vivo affinity was 0.568 nM (95 CI: 0.455-0.710). Estimated degradation (target turnover) rate of free OSM was 2.05 hr⁻¹ (1.62-2.59). Approximately 90% target engagement was estimated in serum following a single 6 mg/kg SC administration of mab 1.

The relationship between plasma concentrations of mab 1 and serum OSM was evaluated as well as the target engagement. In addition, the relationship between plasma mab 1 concentrations and platelet counts was evaluated and the results showed a potential impact on thrombocytopenia and anaemia. The levels were within the required safety margins. Having determined the safe and efficacious single dose it was then necessary to determine what the repeat dosing level necessary for required TE would be.

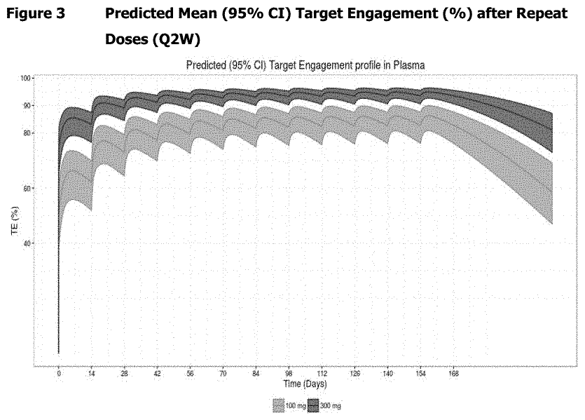

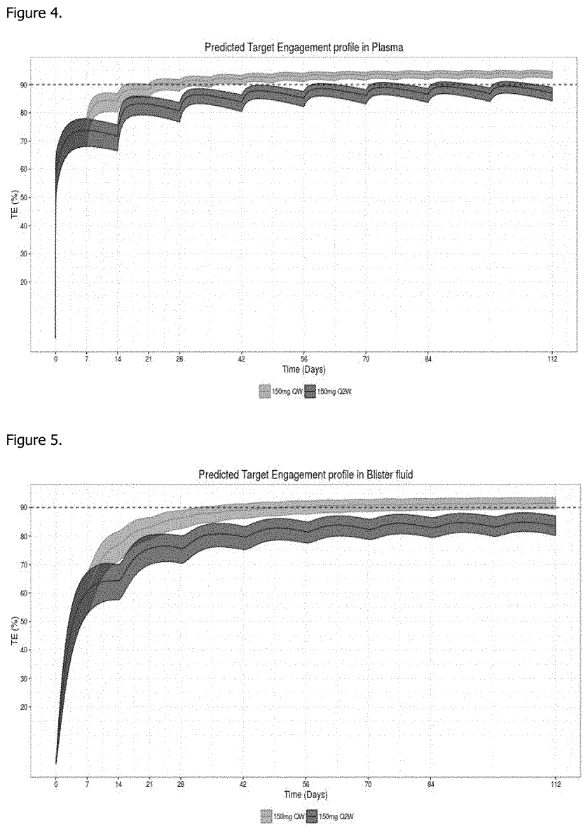

FIG. 3 illustrates the simulated mAb1 TE profile during repeat dosing based on the one compartment PK-TE model. According to the model, a dose of 100 mg SC every other week is predicted to achieve sub-maximal TE (approximately 80% at steady state trough levels), while 300 mg SC every other week is predicted to achieve TE above 90%. Hence a 150 mg dose is expected to achieve necessary target engagement, see FIGS. 4 and 5 in a single more concentrated administration as per Table 4.

TABLE 4

| | |
|---|---|
| Dosage formulation: | Solution for injection; 50 |
| | mM sodium acetate, |
| | 0.05 mM EDTA, 1.0% |
| | arginine, 51 mM sodium |
| | chloride, pH 5.5 with |
| | 0.02% polysorbate 80 |
| Unit dose | 1.2 mL fill with 1 mL |
| strength(s)/Dosage | extractable volume at |
| level(s): | 150 mg/mL |
| Route of | SC injection only |
| Administration | |
| Dosing instructions: | Future study 150 mg dose |
| | 1 × 1 ml injected via |
| | needle and syringe |

The 150 mg dose is predicted to reach the required TE when administered weekly. However, a 150 mg dose given every 2 weeks may reach sub optimal TE but may be better tolerated with regards to thrombocytopenia and anaemia.

### Example 3: Mab 2 FTIH Phase I Study

A previous clinical study using mab 2 was done to assess safety, tolerability, efficacy, pharmacokinetics and pharmacodynamics of single (Part A) and 3 repeat (Part B) intravenous infusions in patients with active RA on a background of methotrexate (MTX). Mab 2 has a heavy chain sequence of SEQ ID NO: 47 and a light chain sequence of SEQ ID NO: 48.

Part C was a single dose, randomised, single-blind, placebo-controlled study to assess subcutaneously administered mab 2 to patients with active RA on a background of MTX. Patients in Cohorts 1 through 6 received 0.03 mg/kg, 0.3 mg/kg, 3 mg/kg (2 cohorts of patients were enrolled at this dose level), 10 mg/kg and 30 mg/kg of mab 2; doses were administered in a dose escalation. Cohorts 2 through 6 were dosed a minimum of three weeks after dosing of the last patient in the previous cohort. Cohorts 7 and 8 enrolled simultaneously, and patients received 10 mg/kg or 20 mg/kg mab 2.

Part B was a randomized, double-blind, placebo-controlled, repeat dose study based on changes in Disease Activity Score 28 (DAS28) and PK in Part A. Prior to administration of the first dose, eligible patients (n=54) were randomized in a 2:1 ratio to receive mab 2 (n=37) or placebo (n=17). For each patient, doses were administered approximately four weeks apart.

In Parts A and B, mab 2 or placebo was administered by slow IV infusion over two hours. The primary endpoint of the study was mean change in DAS28 at Day 28 in Part A and Day 56 in Part B and C. All patients receiving at least one dose of mab 2 were included in safety analysis. In Part A, there were statistically significant differences in DAS28 between 3 mg/kg and placebo at Day 56, 84 and 91. There was also a statistically significant difference in DAS28 between 0.3 mg/kg, 3 mg/kg and 10 mg/kg, as compared to placebo, at Day 84. Although these changes were small and occurred late, they supported progression to Part B and C to determine the therapeutic potential of mab 2. For Part B, no significant difference was observed between 6 mg/kg and placebo. For Part C, a statistically significant difference in DAS28 was observed at Day 40, Day 84 and Day 100 between the 500 mg subcutaneous group, as compared to placebo. No significant findings were observed at any of the time points for European League Against Rheumatism (EULAR) response criteria, ACR20, ACR50 or ACR70. An exploratory analysis of clinical, pharmacokinetic and pharmacodynamics data suggests the lack of efficacy may be due to the binding affinity and rapid off-rate of mab 2 as compared to the higher affinity OSM receptor causing a protein carrier effect prolonging the half-life of OSM due to accumulation of the OSM/antibody complex in the serum and synovial fluid.

Our data highlighted the importance of binding affinity and off-rate effect of a mAb to fully neutralize the target and how this may influence its efficacy and potentially worsen disease activity.

The improved affinity of the 10G8 antibody, and humanised mab 1 derived from 10G8, resulted in a similar platelet effect (proof of pharmacology) at lower doses with respect to mab 2.

TABLE 5

| | | Percentage decrease in platelet number of patients (%) | | | | | |
|---|---|---|---|---|---|---|---|
| Adverse events - number of patients (%) | | Any | Any AE | 25 to | 39 to | 52 to | |
| Part | Treatment group (N) | AE | (days 1-28) | 38% | 51% | 85% | All |
| A | 0.03/0.06 mg/kg IV (N = 4)^{a} | 2 (50) | 1 (25) | 0 | 0 | 0 | 0 |
| | 0.3 mg/kg IV (N = 8)^{a} | 2 (25) | 2 (25) | 1 (13%) | 0 | 0 | 1 (13%) |
| | 3 mg/kg IV (N = 12) | 7 (58) | 5 (42) | 6 (50%) | 0 | 0 | 6 (50%) |
| | 10 mg/kg IV (N = 12) | 6 (50) | 5 (42) | 4 (33%) | 0 | 1 (8%) | 5 (42%) |
| | 20 mg/kg IV (N = 6) | 5 (83) | 4 (67) | 2 (33%) | 2 (33%) | 0 | 4 (67%) |
| | 30 mg/kg IV (N = 6) | 4 (67) | 3 (50) | 1 (17%) | 2 (33%) | 0 | 3 (50%) |
| B | 6 mg/kg repeat IV (N = 37) | 15 (41) | 8 (22) | 10 (27%) | 4 (11%) | 1 (3%) | 15 (41%) |
| C | 500 mg SC (N = 12) | 6 (50) | 6 (50) | 5 (42%) | 2 (17%) | 0 | 7 (58%) |
| Pooled (Parts A, | All Placebo (N = 38) | 12 (32) | 8 (21) | 8 (21%) | 1 (3%) | 1 (3%) | 10 (26%) |
| B, C) | | | | | | | |

AE, adverse event; IV, intravenous patient; SC, subcutaneous patient.

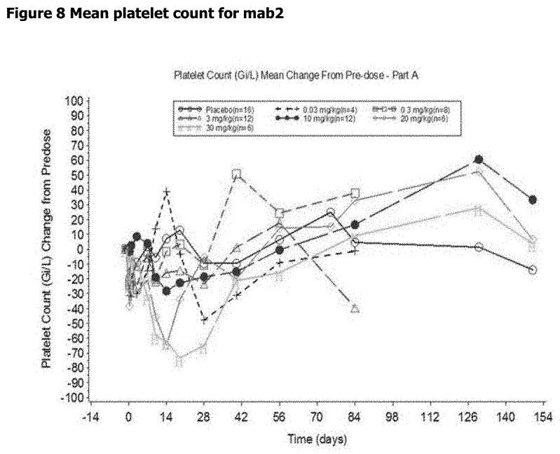

There was a dose related decrease in platelet number. Percentage and mean changes from baseline are detailed in Table 5 and FIG. 8, respectively.

This decrease in platelet count is consistent with the pharmacology of mab 2 and appeared to be dose proportional with platelets demonstrating a greater decrease from baseline over a longer period of time.

From Table 5, a dosage of 3 mg/kg gave a reduction of between 28 to 35% in 50% of the subjects, that means that the mean effect is definitively lower than 50%, approx. between 10-20%. FIG. 8 shows a similar percentage change. A dosage of 10 mg/kg showed a 20-30% reduction, a 20 mg/kg dosage a 50-60%, reduction and a 30 mg/kg dosage a 60-70% reduction. All doses for mab 2 were carried out using an IV route of administration.

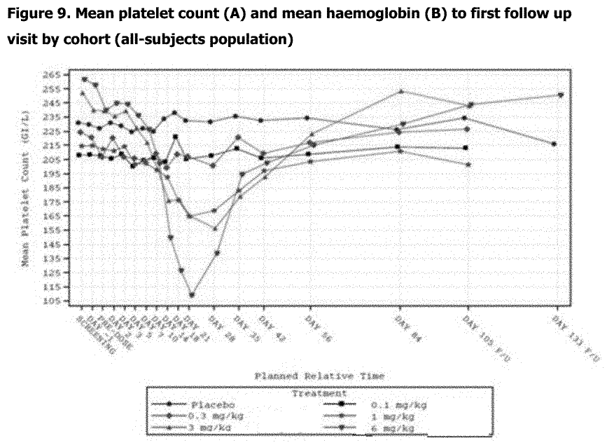

In comparison mab 1 was assessed for platelet reduction in a recent FTIH study following a protocol as outlined in Example 1 (not prophetic data). FIG. 9 shows that a 3 mg/kg (SC) dosage in said FTIH mab1 study produced a 35% reduction and a 6 mg/kg (SC) dosage a 60% reduction. Assuming an 80% bioavailability, there is a 5 to 6 fold ratio potency (based on platelet effect) between mab 1 and mab 2. Our data highlight the importance of binding affinity and off-rate effect of a mAb to fully neutralize the target and based on this how the dosage regimen may influence its efficacy.

Sequence Summary (Table A)

| | | Polynucleotide |
|---|---|---|
| Description | Amino acid sequence | sequence |
| 10G8 CDRH1 | SEQ. I.D. NO: 1 | n/a |
| 10G8 CDRH2 | SEQ. I.D. NO: 2 | n/a |
| 10G8 CDRH3 | SEQ. I.D. NO: 3 | n/a |
| 10G8 CDRL1 | SEQ. I.D. NO: 4 | n/a |
| 10G8 CDRL2 | SEQ. I.D. NO: 5 | n/a |
| 10G8 CDRL3 | SEQ. I.D. NO: 6 | n/a |
| 10G8 V_{H} domain (murine) | SEQ. I.D. NO: 8 | SEQ. I.D. NO: 7 |
| 10G8 V_{L} domain (murine) | SEQ. I.D. NO: 10 | SEQ. I.D. NO: 9 |
| 10G8 V_{H} domain (chimera) | SEQ. I.D. NO: 12 | SEQ. I.D. NO: 11 |
| 10G8 V_{L} domain (chimera) | SEQ. I.D. NO: 14 | SEQ. I.D. NO: 13 |
| IGHV3_7 human variable heavy chain | SEQ. I.D. NO: 16 | SEQ. I.D. NO: 15 |
| germ line acceptor nucleotide | | |
| sequence | | |
| IGKV4_1 human variable light chain | SEQ. I.D. NO: 18 | SEQ. I.D. NO: 17 |
| germ line acceptor nucleotide | | |
| sequence | | |
| 10G8 Humanised V_{H} H0 (nucleotide | SEQ. I.D. NO: 20 | SEQ. I.D. NO: 19 |
| sequence was leto codon optimised) | | |
| 10G8 Humanised V_{H} H1 (nucleotide | SEQ. I.D. NO: 22 | SEQ. I.D. NO: 21 |
| sequence was leto codon optimised) | | |
| 10G8 Humanised V_{H} H2 (nucleotide | SEQ. I.D. NO: 24 | SEQ. I.D. NO: 23 |
| sequence was leto codon optimised) | | |
| 10G8 Humanised V_{L} L0 (nucleotide | SEQ. I.D. NO: 26 | SEQ. I.D. NO: 25 |
| sequence was leto codon optimised) | | |
| 10G8 Humanised V_{L} L1 (nucleotide | SEQ. I.D. NO: 28 | SEQ. I.D. NO: 27 |
| sequence was leto codon optimised) | | |
| 10G8 Humanised V_{L} L2 (nucleotide | SEQ. I.D. NO: 30 | SEQ. I.D. NO: 29 |
| sequence was leto codon optimised) | | |
| 10G8 Humanised V_{L} L3 (nucleotide | SEQ. I.D. NO: 32 | SEQ. I.D. NO: 31 |
| sequence was leto codon optimised) | | |
| 10G8 Humanised V_{L} L4 (nucleotide | SEQ. I.D. NO: 34 | SEQ. I.D. NO: 33 |
| sequence was leto codon optimised) | | |
| Mature H0 heavy chain (nucleotide | SEQ. I.D. NO: 36 | SEQ. I.D. NO: 35 |
| sequence was leto codon optimised) | | |
| Mature L1 light chain (nucleotide | SEQ. I.D. NO: 38 | SEQ. I.D. NO: 37 |
| sequence was leto codon optimised) | | |
| Humanised VH variant H0 (IGHV3_23 | SEQ. I.D. NO: 40 | SEQ. I.D. NO: 39 |
| CDRH1) (nucleotide sequence was | | |
| leto codon optimised) | | |
| Mature H0 (IGHV3_23 CDRH1) heavy | SEQ. I.D. NO: 42 | SEQ. I.D. NO: 41 |
| chain (nucleotide sequence was leto | | |
| codon optimised) | | |
| Human heavy chain germline | SEQ. I.D. NO: 43 | n/a |
| IGHV3_23 CDRH1 | | |
| Human light chain germline IGKV1_5 | SEQ. I.D. NO: 44 | n/a |
| CDRL2 | | |
| Human OSM | SEQ. I.D. NO: 46 | SEQ. I.D. NO: 45 |
| mAb 2 Heavy chain | SEQ. I.D. NO: 47 | n/a |
| mAb 2 Light chain | SEQ. I.D. NO: 48 | n/a |

Sequence Listing
10G8 CDRH1
SEQ ID NO: 1
NYAMS
10G8 CDRH2
SEQ ID NO: 2
TISDGGSFTYYLDNVRG
10G8 CDRH3
SEQ ID NO: 3
DVGHTTFWYFDV
10G8 CDRL1
SEQ ID NO: 4
RASKSVSAAGYNFMH
10G8 CDRL2
SEQ ID NO: 5
YASNLES
10G8 CDRL3
SEQ ID NO: 6
LHSREFPFT
10G8 V_{H} nucleotide sequence
SEQ ID NO: 7
GAAATGCAACTGGTGGAGTCTGGGGAAGGCTTAGTGGAGCCTGGAGGGTCCCTGAAACTCTCC
TGTGCAGCCTCTGGATTCACTTTCAGTAACTATGCCATGTCTTGGGTTCGCCAGACTCCGGAA
AAGAGCCTGGAGTGGGTCGCAACCATTAGTGATGGTGGTAGTTTCACCTACTATCTAGACAAT
GTAAGGGGCCGATTCACCATCTCCAGAGACAATGCCAAGAACAACCTGTATTTGCAAATGAGC
CATTTGAAGTCTGACGACACAGCCATGTATTACTGTGCAAGAGATGTGGGACATACTACCTTT
TGGTACTTCGATGTCTGGGGCTCAGGGACCGCGGTCACCGTCTCCTCA
10G8 V_{H} amino acid sequence
SEQ ID NO: 8
EMQLVESGEGLVEPGGSLKLSCAASGFTFSNYAMSVVVRQTPEKSLEWVATISDGGSFTYYLD
NVRGRFTISRDNAKNNLYLQMSHLKSDDTAMYYCARDVGHTTFWYFDVWGSGTAVTVSS
10G8 V_{L} nucleotide sequence
SEQ ID NO: 9
GACATTGTGCTGACACAGTCTCCTGTTTTCTTAGTTGTATCTCTGGGGCAGAGGGCCACCATC
TCCTGTAGGGCCAGCAAAAGTGTCAGTGCAGCTGGCTATAATTTCATGCACTGGTACCAACAG
AAACCAGGACAGCCGCCCAAAGTCCTCATCAAGTATGCATCCAACCTAGAATCTGGGGTCCCT
GCCAGGTTCAGTGGCAGTGGGTCTGGGACAGACTTCACCCTCAACATCCATCCTGTGGAGGAG
GAGGATGCTGTAACATATTACTGTCTGCACAGTAGGGAGTTTCCGTTCACGTTCGGAGGGGGG
ACCAACCTGGAAATAAAA
10G8 V_{L} amino acid sequence
SEQ ID NO: 10
DIVLTQSPVFLVVSLGQRATISCRASKSVSAAGYNFMHWYQQKPGQPPKVLIKYASNLESGVP
ARFSGSGSGTDFTLNIHPVEEEDAVTYYCLHSREFPFTFGGGTNLEIK
10G8 V_{H} chimera nucleotide sequence
SEQ ID NO: 11
GAAATGCAACTGGTGGAGTCTGGGGAAGGCTTAGTGGAGCCTGGAGGGTCCCTGAAACTCTCC
TGTGCAGCCTCTGGATTCALTTTCAGTAACTATGCCATGTCTTGGGTTCGCCAGACTCCGGAA
AAGAGCCTGGAGTGGGTCGCAACCATTAGTGATGGTGGTAGTTTCACCTACTATCTAGACAAT
GTAAGGGGCCGATTCACCATCTCCAGAGACAATGCCAAGAACAACCTGTATTTGCAAATGAGC
CATTTGAAGTCTGACGACACAGCCATGTATTACTGTGCAAGAGATGTGGGACATACTACCTTT
TGGTACTTCGATGTCTGGGGCTCAGGGACACTAGTGACCGTGTCCAGCGCCAGCACCAAGGGC
CCCAGCGTGTTCCCCCTGGCCCCCAGCAGCAAGAGCACCAGCGGCGGCACAGCCGCCCTGGGC
TGCCTGGTGAAGGACTACTTCCCCGAACCGGTGACCGTGTCCTGGAACAGCGGAGCCCTGACC
AGCGGCGTGCACACCTTCCCCGCCGTGCTGCAGAGCAGCGGCCTGTACAGCCTGAGCAGCGTG
GTGACCGTGCCCAGCAGCAGCCTGGGCACCCAGACCTACATCTGTAACGTGAACCACAAGCCC
AGCAACACCAAGGTGGACAAGAAGGTGGAGCCCAAGAGCTGTGACAAGACCCACACCTGCCCC
CCCTGCCCTGCCCCCGAGCTGCTGGGAGGCCCCAGCGTGTTCCTGTTCCCCCCCAAGCCTAAG
GACACCCTGATGATCAGCAGAACCCCCGAGGTGACCTGTGTGGTGGTGGATGTGAGCCACGAG
GACCCTGAGGTGAAGTTCAACTGGTACGTGGACGGCGTGGAGGTGCACAATGCCAAGACCAAG
CCCAGGGAGGAGCAGTACAACAGCACCTACCGGGTGGTGTCCGTGCTGACCGTGCTGCACCAG
GATTGGCTGAACGGCAAGGAGTACAAGTGTAAGGTGTCCAACAAGGCCCTGCCTGCCCCTATC
GAGAAAACCATCAGCAAGGCCAAGGGCCAGCCCAGAGAGCCCCAGGTGTACACCCTGCCCCCT
AGCAGAGATGAGCTGACCAAGAACCAGGTGTCCCTGACCTGCCTGGTGAAGGGCTTCTACCCC
AGCGACATCGCCGTGGAGTGGGAGAGCAACGGCCAGCCCGAGAACAACTACAAGACCACCCCC
CCTGTGCTGGACAGCGATGGCAGCTTCTTCCTGTACAGCAAGCTGACCGTGGACAAGAGCAGA
TGGCAGCAGGGCAACGTGTTCAGCTGCTCCGTGATGCACGAGGCCCTGCACAATCACTACACC
CAGAAGAGCCTGAGCCTGTCCCCTGGCAAG
10G8 V_{H} chimera amino acid sequence
SEQ ID NO: 12
EMQLVESGEGLVEPGGSLKLSCAASGFTFSNYAMSVVVRQTPEKSLEWVATISDGGSFTYYLD
NVRGRFTISRDNAKNNLYLQMSHLKSDDTAMYYCARDVGHTTFWYFDVWGSGTLVTVSSASTK
GPSVFPLAPSSKSTSGGTAALGCLVKDYFPEPVIVSWNSGALTSGVHTFPAVLQSSGLYSLSS
VVTVPSSSLGTQTYICNVNHKPSNTKVDKKVEPKSCDKTHTCPPCPAPELLGGPSVFLFPPKP
KDTLMISRTPEVTCVVVDVSHEDPEVKFNVVYVDGVEVHNAKTKPREEQYNSTYRVVSVLIVL
HQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLIVDKSRWQQGNVFSCSVMHEALHNH
YTQKSLSLSPGK
10G8 V_{L} chimera nucleotide sequence
SEQ ID NO: 13
GACATTGTGCTGACACAGTCTCCTGTTTTCTTAGTTGTATCTCTGGGGCAGAGGGCCACCATC
TCCTGTAGGGCCAGCAAAAGTGTCAGTGCAGCTGGCTATAATTTCATGCACTGGTACCAACAG
AAACCAGGACAGCCGCCCAAAGTCCTCATCAAGTATGCATCCAACCTAGAATCTGGGGTCCCT
GCCAGGTTCAGTGGCAGTGGGTCTGGGACAGACTTCACCCTCAACATCCATCCTGTGGAGGAG
GAGGATGCTGTAACATATTACTGTCTGCACAGTAGGGAGTTTCCGTTCACGTTCGGAGGGGGG
ACCAACCTGGAAATAAAACGTACGGTGGCCGCCCCCAGCGTGTTCATCTTCCCCCCCAGCGAT
GAGCAGCTGAAGAGCGGCACCGCCAGCGTGGTGTGTCTGCTGAACAACTTCTACCCCCGGGAG
GCCAAGGTGCAGTGGAAGGTGGACAATGCCCTGCAGAGCGGCAACAGCCAGGAGAGCGTGACC
GAGCAGGACAGCAAGGACTCCACCTACAGCCTGAGCAGCACCCTGACCCTGAGCAAGGCCGAC
TACGAGAAGCACAAGGTGTACGCCTGTGAGGTGACCCACCAGGGCCTGTCCAGCCCCGTGACC
AAGAGCTTCAACCGGGGCGAGTGC
10G8 V_{L} chimera amino acid sequence
SEQ ID NO: 14
DIVLTQSPVFLVVSLGQRATISCRASKSVSAAGYNFMHWYQQKPGQPPKVLIKYASNLESGVP
ARFSGSGSGTDFTLNIHPVEEEDAVTYYCLHSREFPFTFGGGTNLEIKRIVAAPSVFIFPPSD
EQLKSGTASVVCLLNNFYPREAKVQWKVDNALQSGNSQESVTEQDSKDSTYSLSSTLTLSKAD
YEKHKVYACEVTHQGLSSPVTKSFNRGEC
IGHV3_7 human V_{H} germline acceptor nucleotide sequence
SEQ ID NO: 15
GAGGTGCAGCTGGTGGAGTCTGGGGGAGGCTTGGTCCAGCCTGGGGGGTCCCTGAGACTCTCC
TGTGCAGCCTCTGGATTCACCTTTAGTAGCTATTGGATGAGCTGGGTCCGCCAGGCTCCAGGG
AAGGGGCTGGAGTGGGTGGCCAACATAAAGCAAGATGGAAGTGAGAAATACTATGTGGACTCT
GTGAAGGGCCGATTCACCATCTCCAGAGACAACGCCAAGAACTCACTGTATCTGCAAATGAAC
AGCCTGAGAGCCGAGGACACGGCTGTGTATTACTGTGCGAGA
IGHV3_7 human V_{H} germline acceptor amino acid sequence
SEQ ID NO: 16
EVQLVESGGGLVQPGGSLRLSCAASGFTFSSYWMSWVRQAPGKGLEWVANIKQDGSEKYYVDS
VKGRFTISRDNAKNSLYLQMNSLRAEDTAVYYCAR
IGKV4_1 human V_{L} germline acceptor nucleotide sequence
SEQ ID NO: 17
GACATCGTGATGACCCAGTCTCCAGACTCCCTGGCTGTGTCTCTGGGCGAGAGGGCCACCATC
AACTGCAAGTCCAGCCAGAGTGTTTTATACAGCTCCAACAATAAGAACTACTTAGCTTGGTAC
CAGCAGAAACCAGGACAGCCTCCTAAGCTGCTCATTTACTGGGCATCTACCCGGGAATCCGGG
GTCCCTGACCGATTCAGTGGCAGCGGGTCTGGGACAGATTTCACTCTCACCATCAGCAGCCTG
CAGGCTGAAGATGTGGCAGTTTATTACTGTCAGCAATATTATAGTACT
IGKV4_1 human V_{L} germline acceptor amino acid sequence
SEQ ID NO: 18
DIVMTQSPDSLAVSLGERATINCKSSQSVLYSSNNKNYLAVVYQQKPGQPPKLLIYWASTRES
GVPDRFSGSGSGTDFTLTISSLQAEDVAVYYCQQYYST
10G8 Humanised V_{H} H0 nucleotide sequence - leto codon
optimised
SEQ ID NO: 19
GAGGTGCAGCTGGTGGAAAGCGGCGGCGGCCTGGTCCAGCCCGGCGGGAGCCTGAGACTCTCT
TGCGCCGCTAGCGGCTTCACCTTCAGCAACTACGCCATGAGCTGGGTGAGGCAGGCCCCCGGC
AAGGGCCTGGAGTGGGTGGCCACCATCAGCGACGGCGGCAGCTTCACCTACTATCTGGACAAC
GTGAGGGGCAGGTTCACCATCAGCAGGGACAACGCCAAGAACAGCCTGTACCTGCAGATGAAC
AGCCTGAGGGCCGAGGATACCGCCGTGTACTACTGCGCCAGGGACGTCGGCCACACCACCTTC
TGGTACTTCGACGTCTGGGGCAGGGGCACACTAGTGACCGTGTCCAGC
10G8 Humanised V_{H} H0 amino acid sequence
SEQ ID NO: 20
EVQLVESGGGLVQPGGSLRLSCAASGFTFSNYAMSWVRQAPGKGLEWVATISDGGSFTYYLDN
VRGRFTISRDNAKNSLYLQMNSLRAEDTAVYYCARDVGHTTFVVYFDVWGRGTLVTVSS
10G8 Humanised V_{H} H1 nucleotide sequence - leto codon
optimised
SEQ ID NO: 21
GAGATGCAGCTGGTGGAAAGCGGCGGCGGCCTGGTCCAGCCCGGCGGGAGCCTGAGACTCTCT
TGCGCCGCTAGCGGCTTCACCTTCAGCAACTACGCCATGAGCTGGGTGAGGCAGGCCCCCGGC
AAGGGCCTGGAGTGGGTGGCCACCATCAGCGACGGCGGCAGCTTCACCTACTATCTGGACAAC
GTGAGGGGCAGGTTCACCATCAGCAGGGACAACGCCAAGAACAGCCTGTACCTGCAGATGAAC
AGCCTGAGGGCCGAGGATACCGCCGTGTACTACTGCGCCAGGGACGTCGGCCACACCACCTTC
TGGTACTTCGACGTCTGGGGCAGGGGCACACTAGTGACCGTGTCCAGC
10G8 Humanised V_{H} H1 amino acid sequence
SEQ ID NO: 22
EMQLVESGGGLVQPGGSLRLSCAASGFTFSNYAMSWVRQAPGKGLEWVATISDGGSFTYYLDN
VRGRFTISRDNAKNSLYLQMNSLRAEDTAVYYCARDVGHTTFWYFDVWGRGTLVTVSS
10G8 Humanised V_{H} H2 nucleotide sequence - leto codon
optimised
SEQ ID NO: 23
GAGGTGCAGCTGGTGGAAAGCGGCGGCGGCCTGGTCCAGCCCGGCGGGAGCCTGAGACTCTCT
TGCGCCGCTAGCGGCTTCACCTTCAGCAACTACGCCATGAGCTGGGTGAGGCAGGCCCCCGGC
AAGGGCCTGGAGTGGGTGGCCACCATCAGCGACGGCGGCAGCTTCACCTACTATCTGGACAAC
GTGAGGGGCAGGTTCACCATCAGCAGGGACAACGCCAAGAACAGCCTGTACCTGCAGATGAAC
AGCCTGAGGGCCGAGGATACCGCCGTGTACTACTGCGCCAGGGACGTCGGCCACACCACCTTC
TGGTACTTCGACGTCTGGGGCTCCGGCACACTAGTGACCGTGTCCAGC
10G8 Humanised V_{H} H2 amino acid sequence
SEQ ID NO: 24
EVQLVESGGGLVQPGGSLRLSCAASGFTFSNYAMSWVRQAPGKGLEWVATISDGGSFTYYLDN
VRGRFTISRDNAKNSLYLQMNSLRAEDTAVYYCARDVGHTTFVVYFDVWGSGTLVTVSS
10G8 Humanised V_{L} L0 nucleotide sequence - leto codon
optimised
SEQ ID NO: 25
GACATCGTGATGACTCAGAGCCCCGATAGCCTGGCCGTGAGCCTGGGCGAAAGGGCCACCATC
AACTGCAGGGCCAGCAAGAGCGTGAGCGCTGCCGGCTACAACTTCATGCACTGGTACCAGCAG
AAGCCCGGCCAGCCCCCCAAGCTGCTGATCTACTACGCCTCCAACCTGGAGAGCGGCGTGCCA
GACAGGTTCAGCGGATCTGGCAGCGGCACCGACTTCACCCTGACCATCTCAAGCCTGCAGGCC
GAGGACGTCGCCGTGTACTACTGCCTGCACAGCAGGGAGTTCCCCTTCACCTTTGGCGGCGGC
ACCAAGGTGGAGATCAAG
10G8 Humanised V_{L} L0 amino acid sequence
SEQ ID NO: 26
DIVMTQSPDSLAVSLGERATINCRASKSVSAAGYNFMHWYQQKPGQPPKLLIYYASNLESGVP
DRFSGSGSGTDFTLTISSLQAEDVAVYYCLHSREFPFTFGGGTKVEIK
10G8 Humanised V_{L} L1 nucleotide sequence - leto codon
optimised
SEQ ID NO: 27
GACATCGTGATGACTCAGAGCCCCGATAGCCTGGCCGTGAGCCTGGGCGAAAGGGCCACCATC
AACTGCAGGGCCAGCAAGAGCGTGAGCGCTGCCGGCTACAACTTCATGCACTGGTACCAGCAG
AAGCCCGGCCAGCCCCCCAAGGTGCTGATCTACTACGCCTCCAACCTGGAGAGCGGCGTGCCA
GACAGGTTCAGCGGATCTGGCAGCGGCACCGACTTCACCCTGACCATCTCAAGCCTGCAGGCC
GAGGACGTCGCCGTGTACTACTGCCTGCACAGCAGGGAGTTCCCCTTCACCTTTGGCGGCGGC
ACCAAGGTGGAGATCAAG
10G8 Humanised V_{L} L1 amino acid sequence
SEQ ID NO: 28
DIVMTQSPDSLAVSLGERATINCRASKSVSAAGYNFMHVVYQQKPGQPPKVLIYYASNLESGV
PDRFSGSGSGTDFTLTISSLQAEDVAVYYCLHSREFPFTFGGGTKVEIK
10G8 Humanised V_{L} L2 nucleotide sequence- leto codon
optimised
SEQ ID NO: 29
GACATCGTGATGACTCAGAGCCCCGATAGCCTGGCCGTGAGCCTGGGCGAAAGGGCCACCATC
AACTGCAGGGCCAGCAAGAGCGTGAGCGCTGCCGGCTACAACTTCATGCACTGGTACCAGCAG
AAGCCCGGCCAGCCCCCCAAGCTGCTGATCTACTACGCCTCCAACCTGGAGAGCGGCGTGCCA
GACAGGTTCAGCGGATCTGGCAGCGGCACCGACTTCACCCTGACCATCTCAAGCCTGCAGGCC
GAGGACGTCGTGGTGTACTACTGCCTGCACAGCAGGGAGTTCCCCTTCACCTTTGGCGGCGGC
ACCAAGGTGGAGATCAAG
10G8 Humanised V_{L} L2 amino acid sequence
SEQ ID NO: 30
DIVMTQSPDSLAVSLGERATINCRASKSVSAAGYNFMHWYQQKPGQPPKLLIYYASNLESGVP
DRFSGSGSGTDFTLTISSLQAEDVVVYYCLHSREFPFTFGGGTKVEIK
10G8 Humanised V_{L} L3 nucleotide sequence - leto codon
optimised
SEQ ID NO: 31
GACATCGTGATGACTCAGAGCCCCGATAGCCTGGCCGTGAGCCTGGGCGAAAGGGCCACCATC
AACTGCAGGGCCAGCAAGAGCGTGAGCGCTGCCGGCTACAACTTCATGCACTGGTACCAGCAG
AAGCCCGGCCAGCCCCCCAAGCTGCTGATCTACTACGCCTCCAACCTGGAGAGCGGCGTGCCA
GACAGGTTCAGCGGATCTGGCAGCGGCACCGACTTCACCCTGACCATCTCAAGCCTGCAGGCC
GAGGACGTCGCCGTGTACTACTGCCTGCACAGCAGGGAGTTCCCCTTCACCTTTGGCGGCGGC
ACCAACGTGGAGATCAAG
10G8 Humanised V_{L} L3 amino acid sequence
SEQ ID NO: 32
DIVMTQSPDSLAVSLGERATINCRASKSVSAAGYNFMHWYQQKPGQPPKLLIYYASNLESGVP
DRFSGSGSGTDFTLTISSLQAEDVAVYYCLHSREFPFTFGGGTNVEIK
10G8 Humanised V_{L} L4 nucleotide sequence - leto codon
optimised
SEQ ID NO: 33
GACATCGTGATGACTCAGAGCCCCGATAGCCTGGCCGTGAGCCTGGGCGAAAGGGCCACCATC
AACTGCAGGGCCAGCAAGAGCGTGAGCGCTGCCGGCTACAACTTCATGCACTGGTACCAGCAG
AAGCCCGGCCAGCCCCCCAAGGTGCTGATCTACTACGCCTCCAACCTGGAGAGCGGCGTGCCA
GACAGGTTCAGCGGATCTGGCAGCGGCACCGACTTCACCCTGACCATCTCAAGCCTGCAGGCC
GAGGACGTCGTGGTGTACTACTGCCTGCACAGCAGGGAGTTCCCCTTCACCTTTGGCGGCGGC
ACCAACGTGGAGATCAAG
10G8 Humanised V_{L} L4 amino acid sequence
SEQ ID NO: 34
DIVMTQSPDSLAVSLGERATINCRASKSVSAAGYNFMHVVYQQKPGQPPKVLIYYASNLESGV
PDRFSGSGSGTDFTLTISSLQAEDVVVYYCLHSREFPFTFGGGTNVEIK
Mature H0 heavy chain nucleotide sequence - leto codon
optimised
SEQ ID NO: 35
GAGGTGCAGCTGGTGGAAAGCGGCGGCGGCCTGGTCCAGCCCGGCGGGAGCCTGAGACTCTCT
TGCGCCGCTAGCGGCTTCACCTTCAGCAACTACGCCATGAGCTGGGTGAGGCAGGCCCCCGGC
AAGGGCCTGGAGTGGGTGGCCACCATCAGCGACGGCGGCAGCTTCACCTACTATCTGGACAAC
GTGAGGGGCAGGTTCACCATCAGCAGGGACAACGCCAAGAACAGCCTGTACCTGCAGATGAAC
AGCCTGAGGGCCGAGGATACCGCCGTGTACTACTGCGCCAGGGACGTCGGCCACACCACCTTC
TGGTACTTCGACGTCTGGGGCAGGGGCACACTAGTGACCGTGTCCAGCGCCAGCACCAAGGGC
CCCAGCGTGTTCCCCCTGGCCCCCAGCAGCAAGAGCACCAGCGGCGGCACAGCCGCCCTGGGC
TGCCTGGTGAAGGACTACTTCCCCGAACCGGTGACCGTGTCCTGGAACAGCGGAGCCCTGACC
AGCGGCGTGCACACCTTCCCCGCCGTGCTGCAGAGCAGCGGCCTGTACAGCCTGAGCAGCGTG
GTGACCGTGCCCAGCAGCAGCCTGGGCACCCAGACCTACATCTGTAACGTGAACCACAAGCCC
AGCAACACCAAGGTGGACAAGAAGGTGGAGCCCAAGAGCTGTGACAAGACCCACACCTGCCCC
CCCTGCCCTGCCCCCGAGCTGCTGGGAGGCCCCAGCGTGTTCCTGTTCCCCCCCAAGCCTAAG
GACACCCTGATGATCAGCAGAACCCCCGAGGTGACCTGTGTGGTGGTGGATGTGAGCCACGAG
GACCCTGAGGTGAAGTTCAACTGGTACGTGGACGGCGTGGAGGTGCACAATGCCAAGACCAAG
CCCAGGGAGGAGCAGTACAACAGCACCTACCGGGTGGTGTCCGTGCTGACCGTGCTGCACCAG
GATTGGCTGAACGGCAAGGAGTACAAGTGTAAGGTGTCCAACAAGGCCCTGCCTGCCCCTATC
GAGAAAACCATCAGCAAGGCCAAGGGCCAGCCCAGAGAGCCCCAGGTGTACACCCTGCCCCCT
AGCAGAGATGAGCTGACCAAGAACCAGGTGTCCCTGACCTGCCTGGTGAAGGGCTTCTACCCC
AGCGACATCGCCGTGGAGTGGGAGAGCAACGGCCAGCCCGAGAACAACTACAAGACCACCCCC
CCTGTGCTGGACAGCGATGGCAGCTTCTTCCTGTACAGCAAGCTGACCGTGGACAAGAGCAGA
TGGCAGCAGGGCAACGTGTTCAGCTGCTCCGTGATGCACGAGGCCCTGCACAATCACTACACC
CAGAAGAGCCTGAGCCTGTCCCCTGGCAAG
Mature H0 heavy chain amino acid sequence
SEQ ID NO: 36
EVQLVESGGGLVQPGGSLRLSCAASGFTFSNYAMSWVRQAPGKGLEWVATISDGGSFTYYLDN
VRGRFTISRDNAKNSLYLQMNSLRAEDTAVYYCARDVGHTTFVVYFDVWGRGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKIWEPKSCDKTHTCPPCPAPELLGGPSVFLFPPKP
KDTLMISRTPEVTCVVVDVSHEDPEVKFNVVYVDGVEVHNAKTKPREEQYNSTYRVVSVLIVL
HQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLIVDKSRWQQGNVFSCSVMHEALHNH
YTQKSLSLSPGK
Mature L1 light chain nucleotide sequence - leto codon
optimised
SEQ ID NO: 37
GACATCGTGATGACTCAGAGCCCCGATAGCCTGGCCGTGAGCCTGGGCGAAAGGGCCACCATC
AACTGCAGGGCCAGCAAGAGCGTGAGCGCTGCCGGCTACAACTTCATGCACTGGTACCAGCAG
AAGCCCGGCCAGCCCCCCAAGGTGCTGATCTACTACGCCTCCAACCTGGAGAGCGGCGTGCCA
GACAGGTTCAGCGGATCTGGCAGCGGCACCGACTTCACCCTGACCATCTCAAGCCTGCAGGCC
GAGGACGTCGCCGTGTACTACTGCCTGCACAGCAGGGAGTTCCCCTTCACCTTTGGCGGCGGC
ACCAAGGTGGAGATCAAGCGTACGGTGGCCGCCCCCAGCGTGTTCATCTTCCCCCCCAGCGAT
GAGCAGCTGAAGAGCGGCACCGCCAGCGTGGTGTGTCTGCTGAACAACTTCTACCCCCGGGAG
GCCAAGGTGCAGTGGAAGGTGGACAATGCCCTGCAGAGCGGCAACAGCCAGGAGAGCGTGACC
GAGCAGGACAGCAAGGACTCCACCTACAGCCTGAGCAGCACCCTGACCCTGAGCAAGGCCGAC
TACGAGAAGCACAAGGTGTACGCCTGTGAGGTGACCCACCAGGGCCTGTCCAGCCCCGTGACC
AAGAGCTTCAACCGGGGCGAGTGC
Mature L1 light chain amino acid sequence
SEQ ID NO: 38
DIVMTQSPDSLAVSLGERATINCRASKSVSAAGYNFMHVVYQQKPGQPPKVLIYYASNLESGV
PDRFSGSGSGTDFTLTISSLQAEDVAVYYCLHSREFPFTFGGGTKVEIKRTVAAPSVFIFPPS
DEQLKSGTASVVCLLNNFYPREAKVQWKVDNALQSGNSQESVTEQDSKDSTYSLSSTLTLSKA
DYEKHKVYACEVTHQGLSSPVTKSFNRGEC
Humanised V_{H} variant HO (IGHV3_23 CDRH1) nucleotide
sequence - leto codon optimised
SEQ ID NO: 39
GAGGTGCAGCTGGTGGAAAGCGGCGGCGGCCTGGTCCAGCCCGGCGGGAGCCTGAGACTCTCT
TGCGCCGCTAGCGGCTTCACCTTCAGCAGCTACGCCATGAGCTGGGTGAGGCAGGCCCCCGGC
AAGGGCCTGGAGTGGGTGGCCACCATCAGCGACGGCGGCAGCTTCACCTACTATCTGGACAAC
GTGAGGGGCAGGTTCACCATCAGCAGGGACAACGCCAAGAACAGCCTGTACCTGCAGATGAAC
AGCCTGAGGGCCGAGGATACCGCCGTGTACTACTGCGCCAGGGACGTCGGCCACACCACCTTC
TGGTACTTCGACGTCTGGGGCAGGGGCACACTAGTGACCGTGTCCAGC
Humanised V_{H} variant H0 (IGHV3_23 CDRH1) amino acid
sequence
SEQ ID NO: 40
EVQLVESGGGLVQPGGSLRLSCAASGFTFSSYAMSVVVRQAPGKGLEWVATISDGGSFTYYLD
NVRGRFTISRDNAKNSLYLQMNSLRAEDTAVYYCARDVGHTTFWYFDVWGRGTLVTVSS
Mature H0 (IGHV3_23 CDRH1) heavy chain nucleotide
sequence - leto codon optimised
SEQ ID NO: 41
GAGGTGCAGCTGGTGGAAAGCGGCGGCGGCCTGGTCCAGCCCGGCGGGAGCCTGAGACTCTCT
TGCGCCGCTAGCGGCTTCACCTTCAGCAGCTACGCCATGAGCTGGGTGAGGCAGGCCCCCGGC
AAGGGCCTGGAGTGGGTGGCCACCATCAGCGACGGCGGCAGCTTCACCTACTATCTGGACAAC
GTGAGGGGCAGGTTCACCATCAGCAGGGACAACGCCAAGAACAGCCTGTACCTGCAGATGAAC
AGCCTGAGGGCCGAGGATACCGCCGTGTACTACTGCGCCAGGGACGTCGGCCACACCACCTTC
TGGTACTTCGACGTCTGGGGCAGGGGCACACTAGTGACCGTGTCCAGCGCCAGCACCAAGGGC
CCCAGCGTGTTCCCCCTGGCCCCCAGCAGCAAGAGCACCAGCGGCGGCACAGCCGCCCTGGGC
TGCCTGGTGAAGGACTACTTCCCCGAACCGGTGACCGTGTCCTGGAACAGCGGAGCCCTGACC
AGCGGCGTGCACACCTTCCCCGCCGTGCTGCAGAGCAGCGGCCTGTACAGCCTGAGCAGCGTG
GTGACCGTGCCCAGCAGCAGCCTGGGCACCCAGACCTACATCTGTAACGTGAACCACAAGCCC
AGCAACACCAAGGTGGACAAGAAGGTGGAGCCCAAGAGCTGTGACAAGACCCACACCTGCCCC
CCCTGCCCTGCCCCCGAGCTGCTGGGAGGCCCCAGCGTGTTCCTGTTCCCCCCCAAGCCTAAG
GACACCCTGATGATCAGCAGAACCCCCGAGGTGACCTGTGTGGTGGTGGATGTGAGCCACGAG
GACCCTGAGGTGAAGTTCAACTGGTACGTGGACGGCGTGGAGGTGCACAATGCCAAGACCAAG
CCCAGGGAGGAGCAGTACAACAGCACCTACCGGGTGGTGTCCGTGCTGACCGTGCTGCACCAG
GATTGGCTGAACGGCAAGGAGTACAAGTGTAAGGTGTCCAACAAGGCCCTGCCTGCCCCTATC
GAGAAAACCATCAGCAAGGCCAAGGGCCAGCCCAGAGAGCCCCAGGTGTACACCCTGCCCCCT
AGCAGAGATGAGCTGACCAAGAACCAGGTGTCCCTGACCTGCCTGGTGAAGGGCTTCTACCCC
AGCGACATCGCCGTGGAGTGGGAGAGCAACGGCCAGCCCGAGAACAACTACAAGACCACCCCC
CCTGTGCTGGACAGCGATGGCAGCTTCTTCCTGTACAGCAAGCTGACCGTGGACAAGAGCAGA
TGGCAGCAGGGCAACGTGTTCAGCTGCTCCGTGATGCACGAGGCCCTGCACAATCACTACACC
CAGAAGAGCCTGAGCCTGTCCCCTGGCAAG
Mature H0 (IGHV3_23 CDRH1) heavy chain amino acid
sequence
SEQ ID NO: 42
EVQLVESGGGLVQPGGSLRLSCAASGFTFSSYAMSWVRQAPGKGLEWVATISDGGSFTYYLDN
VRGRFTISRDNAKNSLYLQMNSLRAEDTAVYYCARDVGHTTFWYFDVWGRGTLVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPK
DTLMISRTPEVTCVVVDVSHEDPEVKFNVVYVDGVEVHNAKTKPREEQYNSTYRVVSVLIVLH
QDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLIVDKSRWQQGNVFSCSVMHEALHNHY
TQKSLSLSPGK
Human heavy chain germline IGHV3_23 CDRH1
SEQ ID NO: 43
SYAMS
Human light chain germline IGKV1_5 CDRL2
SEQ ID NO: 44
KASSLES
Human OSM polynucleotide sequence
SEQ ID NO: 45
ATGGGGGTACTGCTCACACAGAGGACGCTGCTCAGTCTGGTCCTTGCACTCCTGTTTCCAAGC
ATGGCGAGCATGGCGGCTATAGGCAGCTGCTCGAAAGAGTACCGCGTGCTCCTTGGCCAGCTC
CAGAAGCAGACAGATCTCATGCAGGACACCAGCAGACTCCTGGACCCCTATATACGTATCCAA
GGCCTGGATGTTCCTAAACTGAGAGAGCACTGCAGGGAGCGCCCCGGGGCCTTCCCCAGTGAG
GAGACCCTGAGGGGGCTGGGCAGGCGGGGCTTCCTGCAGACCCTCAATGCCACACTGGGCTGC
GTCCTGCACAGACTGGCCGACTTAGAGCAGCGCCTCCCCAAGGCCCAGGATTTGGAGAGGTCT
GGGCTGAACATCGAGGACTTGGAGAAGCTGCAGATGGCGAGGCCGAACATCCTCGGGCTCAGG
AACAACATCTACTGCATGGCCCAGCTGCTGGACAACTCAGACACGGCTGAGCCCACGAAGGCT
GGCCGGGGGGCCTCTCAGCCGCCCACCCCCACCCCTGCCTCGGATGCTTTTCAGCGCAAGCTG
GAGGGCTGCAGGTTCCTGCATGGCTACCATCGCTTCATGCACTCAGTGGGGCGGGTCTTCAGC
AAGTGGGGGGAGAGCCCGAACCGGAGCCGGAGACACAGCCCCCACCAGGCCCTGAGGAAGGGG
GTGCGCAGGACCAGACCCTCCAGGAAAGGCAAGAGACTCATGACCAGGGGACAGCTGCCCCGG
TAG
Human OSM amino acid sequence
SEQ ID NO: 46
MGVLLTQRTLLSLVLALLFPSMASMAAIGSCSKEYRVLLGQLQKQTDLMQDTSRLLDPYIRIQ
GLDVPKLREHCRERPGAFPSEETLRGLGRRGFLQTLNATLGCVLHRLADLEQRLPKAQDLERS
GLNIEDLEKLQMARPNILGLRNNIYCMAQLLDNSDTAEPTKAGRGASQPPTPTPASDAFQRKL
EGCRFLHGYHRFMHSVGRVFSKWGESPNRSRRHSPHQALRKGVRRTRPSRKGKRLMTRGQLP
R.
mAb 2 Heavy Chain amino acid sequence
SEQ ID NO: 47
EVQLVQSGAEVKKPGASVKVSCKASGYIFTDYNMDVVVRQAPGQKLEWIGDINPNNGGTIDNQ
KFKDRATLTVDKSTSTVYMELSSLRSEDTAVYYCARGIYYYGSHYFDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVLH
QDWLNGKEYKOWSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHYT
QKSLSLSPGK
mAb 2 Light Chain amino acid sequence
SEQ ID NO: 48
EIVLTQSPSSLSASVGDRVTITCSATSSVSVMHWFQKKPGKAPKRWIYDTSKLASGVPSRFSG
SGSGTDYTLTISSLQPEDFATYYCQQWSSNPLTFGGGTKVDIKRTVAAPSVFIFPPSDEQLKS
GTASVVCLLNNFYPREAKVQWKVDNALQSGNSQESVTEQDSKDSTYSLSSTLTLSKADYEKHK
VYACEVTHQGLSSPVTKSFNRGEC

1. A pharmaceutical composition comprising an antigen binding protein, which is capable of binding to OSM and inhibits the binding of OSM to the gp130 receptor, and wherein an effective dose of said pharmaceutical composition comprises 50-300 mg of said antigen binding protein.

2. A pharmaceutical composition according to claim 1, wherein the pharmaceutical composition comprises 100 mg or 150 mg or 200 mg or 300 mg of said antigen binding protein.

3. A pharmaceutical composition according to claim 2, wherein the pharmaceutical composition comprises 100 mg or 150 mg or 300 mg of said antigen binding protein.

4. A pharmaceutical composition according to any preceding claim, wherein the antigen binding protein comprises CDRH3 of SEQ. ID. NO: 3, CDRH2 of SEQ. ID. NO: 2, CDRL1 of SEQ. ID. NO: 4 and CDRL3 of SEQ. ID. NO: 6, and optionally may comprise CDRH1 of SEQ. ID. NO: 1 or SEQ ID NO:43 and CDRL2 of SEQ. ID. NO: 5 or SEQ ID NO: 44.

5. A pharmaceutical composition according to any preceding claim, wherein the antigen binding protein comprises a heavy chain variable region of SEQ. ID. NO: 20 or SEQ ID NO: 40 and a light chain variable region of SEQ. ID. NO:28.

6. A pharmaceutical composition according to any preceding claim, wherein the antigen binding protein comprises a heavy chain according to SEQ ID NO: 42 and a light chain according to SEQ ID NO:38

7. A pharmaceutical composition according to any preceding claim, wherein the antigen binding protein comprises a heavy chain encoded by SEQ ID NO: 41 and a light chain encoded by SEQ ID NO: 37.

8. A pharmaceutical composition according to any preceding claim, wherein the concentration of antigen binding protein within said pharmaceutical composition is 150 mg/ml.

9. A pharmaceutical composition according to any preceding claim, wherein the pharmaceutical composition comprises sodium acetate, EDTA, arginine, sodium chloride Polysorbate 80 and has a pH of 5.5.

10. A pharmaceutical composition according to claim 9, wherein the pharmaceutical composition comprises 50 mM sodium acetate, 0.05 mM EDTA, 1.0% arginine, 51 mM sodium chloride, 0.02% polysorbate 80 and has a pH of 5.5

11. A pharmaceutical composition according to any preceding claim, wherein the composition is to be administered to a human patient.

12. A pharmaceutical composition according to any preceding claim, wherein the composition is to be administered once a week or once every other week or once every month.

13. A pharmaceutical composition according to any preceding claim, wherein the composition is to be administered once every week.

14. A pharmaceutical composition according to any preceding claim, wherein the composition is to be administered subcutaneously.

15. A pharmaceutical composition according to any preceding claim, for use in the treatment of inflammatory or autoimmune disorders or diseases.

16. A pharmaceutical composition according to claim 15, for use in the treatment of Systemic sclerosis.

17. A method of treating a human patient afflicted with an inflammatory or autoimmune disorder or disease which method comprises the step of administering to said patient a pharmaceutical composition according to any one of claims 1-14.

18. Use of a pharmaceutical composition according to any one of claims 1-14, in the manufacture of a medicament for the treatment of inflammatory or autoimmune disorders or diseases.


SEQUENCE LISTING (47 sequences)

SEQ ID NO: 1 - protein, 5 aa, Mus musculus
NYAMS

SEQ ID NO: 2 - protein, 17 aa, Mus musculus
TISDGGSFTYYLDNVRG

SEQ ID NO: 3 - protein, 12 aa, Mus musculus
DVGHTTFWYFDV

SEQ ID NO: 4 - protein, 7 aa, Mus musculus
YASNLES

SEQ ID NO: 5 - protein, 9 aa, Mus musculus
LHSREFPFT

SEQ ID NO: 6 - DNA, 363 bp, Mus musculus
gaaatgcaactggtggagtctggggaaggcttagtggagcctggagggtccctgaaactctcctgtgcagcctctggattcactttcagtaactatgccatgtcttgggttcgccagactccggaaaagagcctggagtgggtcgcaaccattagtgatggtggtagtttcacctactatctagacaatgtaaggggccgattcaccatctccagagacaatgccaagaacaacctgtatttgcaaatgagccatttgaagtctgacgacacagccatgtattactgtgcaagagatgtgggacatactaccttttggtacttcgatgtctggggctcagggaccgcggtcaccgtctcctca

SEQ ID NO: 7 - protein, 121 aa, Mus musculus
EMQLVESGEGLVEPGGSLKLSCAASGFTFSNYAMSWVRQTPEKSLEWVATISDGGSFTYYLDNVRGRFTISRDNAKNNLYLQMSHLKSDDTAMYYCARDVGHTTFWYFDVWGSGTAVTVSS

SEQ ID NO: 8 - DNA, 333 bp, Mus musculus
gacattgtgctgacacagtctcctgttttcttagttgtatctctggggcagagggccaccatctcctgtagggccagcaaaagtgtcagtgcagctggctataatttcatgcactggtaccaacagaaaccaggacagccgcccaaagtcctcatcaagtatgcatccaacctagaatctggggtccctgccaggttcagtggcagtgggtctgggacagacttcaccctcaacatccatcctgtggaggaggaggatgctgtaacatattactgtctgcacagtagggagtttccgttcacgttcggaggggggaccaacctggaaataaaa

SEQ ID NO: 9 - protein, 111 aa, Mus musculus
DIVLTQSPVFLVVSLGQRATISCRASKSVSAAGYNFMHWYQQKPGQPPKVLIKYASNLESGVPARFSGSGSGTDFTLNIHPVEEEDAVTYYCLHSREFPFTFGGGTNLEIK

SEQ ID NO: 10 - DNA, 1352 bp, Artificial
  chimera
aaatgcaactggtggagtctggggaaggcttagtggagcctggagggtccctgaaactctcctgtgcagcctctggattcactttcagtaactatgccatgtcttgggttcgccagactccggaaaagagcctggagtgggtcgcaaccattagtgatggtggtagtttcacctactatctagacaatgtaaggggccgattcaccatctccagagacaatgccaagaacaacctgtatttgcaaatgagccatttgaagtctgacgacacagccatgtattactgtgcaagagatgtgggacatactaccttttggtacttcgatgtctggggctcagggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 11 - protein, 451 aa, Artificial
  chimera
EMQLVESGEGLVEPGGSLKLSCAASGFTFSNYAMSWVRQTPEKSLEWVATISDGGSFTYYLDNVRGRFTISRDNAKNNLYLQMSHLKSDDTAMYYCARDVGHTTFWYFDVWGSGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 654 bp, Artificial
  chimera
gacattgtgctgacacagtctcctgttttcttagttgtatctctggggcagagggccaccatctcctgtagggccagcaaaagtgtcagtgcagctggctataatttcatgcactggtaccaacagaaaccaggacagccgcccaaagtcctcatcaagtatgcatccaacctagaatctggggtccctgccaggttcagtggcagtgggtctgggacagacttcaccctcaacatccatcctgtggaggaggaggatgctgtaacatattactgtctgcacagtagggagtttccgttcacgttcggaggggggaccaacctggaaataaaacgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 13 - protein, 218 aa, Artificial
  chimera
DIVLTQSPVFLVVSLGQRATISCRASKSVSAAGYNFMHWYQQKPGQPPKVLIKYASNLESGVPARFSGSGSGTDFTLNIHPVEEEDAVTYYCLHSREFPFTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 294 bp, Human
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgaga

SEQ ID NO: 15 - protein, 98 aa, Human
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 16 - DNA, 300 bp, Human
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtact

SEQ ID NO: 17 - protein, 100 aa, Human
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYST

SEQ ID NO: 18 - DNA, 363 bp, Artificial
  Humanised VH H0 nucleotide sequence -leto codon optimised
gaggtgcagctggtggaaagcggcggcggcctggtccagcccggcgggagcctgagactctcttgcgccgctagcggcttcaccttcagcaactacgccatgagctgggtgaggcaggcccccggcaagggcctggagtgggtggccaccatcagcgacggcggcagcttcacctactatctggacaacgtgaggggcaggttcaccatcagcagggacaacgccaagaacagcctgtacctgcagatgaacagcctgagggccgaggataccgccgtgtactactgcgccagggacgtcggccacaccaccttctggtacttcgacgtctggggcaggggcacactagtgaccgtgtccagc

SEQ ID NO: 19 - protein, 121 aa, Artificial
  Humanised VH H0 amino acid sequence
EVQLVESGGGLVQPGGSLRLSCAASGFTFSNYAMSWVRQAPGKGLEWVATISDGGSFTYYLDNVRGRFTISRDNAKNSLYLQMNSLRAEDTAVYYCARDVGHTTFWYFDVWGRGTLVTVSS

SEQ ID NO: 20 - DNA, 363 bp, Artificial
  Humanised VH H1 nucleotide sequence -leto codon optimised
gagatgcagctggtggaaagcggcggcggcctggtccagcccggcgggagcctgagactctcttgcgccgctagcggcttcaccttcagcaactacgccatgagctgggtgaggcaggcccccggcaagggcctggagtgggtggccaccatcagcgacggcggcagcttcacctactatctggacaacgtgaggggcaggttcaccatcagcagggacaacgccaagaacagcctgtacctgcagatgaacagcctgagggccgaggataccgccgtgtactactgcgccagggacgtcggccacaccaccttctggtacttcgacgtctggggcaggggcacactagtgaccgtgtccagc

SEQ ID NO: 21 - protein, 121 aa, Artificial
  Humanised VH H1 amino acid sequence
EMQLVESGGGLVQPGGSLRLSCAASGFTFSNYAMSWVRQAPGKGLEWVATISDGGSFTYYLDNVRGRFTISRDNAKNSLYLQMNSLRAEDTAVYYCARDVGHTTFWYFDVWGRGTLVTVSS

SEQ ID NO: 22 - DNA, 363 bp, Artificial
  Humanised VH H2 nucleotide sequence -leto codon optimised
gaggtgcagctggtggaaagcggcggcggcctggtccagcccggcgggagcctgagactctcttgcgccgctagcggcttcaccttcagcaactacgccatgagctgggtgaggcaggcccccggcaagggcctggagtgggtggccaccatcagcgacggcggcagcttcacctactatctggacaacgtgaggggcaggttcaccatcagcagggacaacgccaagaacagcctgtacctgcagatgaacagcctgagggccgaggataccgccgtgtactactgcgccagggacgtcggccacaccaccttctggtacttcgacgtctggggctccggcacactagtgaccgtgtccagc

SEQ ID NO: 23 - protein, 121 aa, Artificial
  Humanised VH H2 amino acid sequence
EVQLVESGGGLVQPGGSLRLSCAASGFTFSNYAMSWVRQAPGKGLEWVATISDGGSFTYYLDNVRGRFTISRDNAKNSLYLQMNSLRAEDTAVYYCARDVGHTTFWYFDVWGSGTLVTVSS

SEQ ID NO: 24 - DNA, 333 bp, Artificial
  Humanised VL L0 nucleotide sequence -leto codon optimised
gacatcgtgatgactcagagccccgatagcctggccgtgagcctgggcgaaagggccaccatcaactgcagggccagcaagagcgtgagcgctgccggctacaacttcatgcactggtaccagcagaagcccggccagccccccaagctgctgatctactacgcctccaacctggagagcggcgtgccagacaggttcagcggatctggcagcggcaccgacttcaccctgaccatctcaagcctgcaggccgaggacgtcgccgtgtactactgcctgcacagcagggagttccccttcacctttggcggcggcaccaaggtggagatcaag

SEQ ID NO: 25 - protein, 111 aa, Artificial
  10G8 Humanised VL L0 amino acid sequence
DIVMTQSPDSLAVSLGERATINCRASKSVSAAGYNFMHWYQQKPGQPPKLLIYYASNLESGVPDRFSGSGSGTDFTLTISSLQAEDVAVYYCLHSREFPFTFGGGTKVEIK

SEQ ID NO: 26 - DNA, 333 bp, Artificial
  Humanised VL L1 nucleotide sequence -leto codon optimised
gacatcgtgatgactcagagccccgatagcctggccgtgagcctgggcgaaagggccaccatcaactgcagggccagcaagagcgtgagcgctgccggctacaacttcatgcactggtaccagcagaagcccggccagccccccaaggtgctgatctactacgcctccaacctggagagcggcgtgccagacaggttcagcggatctggcagcggcaccgacttcaccctgaccatctcaagcctgcaggccgaggacgtcgccgtgtactactgcctgcacagcagggagttccccttcacctttggcggcggcaccaaggtggagatcaag

SEQ ID NO: 27 - protein, 111 aa, Artificial
  Humanised VL L1 amino acid sequence
DIVMTQSPDSLAVSLGERATINCRASKSVSAAGYNFMHWYQQKPGQPPKVLIYYASNLESGVPDRFSGSGSGTDFTLTISSLQAEDVAVYYCLHSREFPFTFGGGTKVEIK

SEQ ID NO: 28 - DNA, 333 bp, Artificial
  Humanised VL L2 nucleotide sequence- leto codon optimised
gacatcgtgatgactcagagccccgatagcctggccgtgagcctgggcgaaagggccaccatcaactgcagggccagcaagagcgtgagcgctgccggctacaacttcatgcactggtaccagcagaagcccggccagccccccaagctgctgatctactacgcctccaacctggagagcggcgtgccagacaggttcagcggatctggcagcggcaccgacttcaccctgaccatctcaagcctgcaggccgaggacgtcgtggtgtactactgcctgcacagcagggagttccccttcacctttggcggcggcaccaaggtggagatcaag

SEQ ID NO: 29 - protein, 111 aa, Artificial
  Humanised VL L2 amino acid sequence
DIVMTQSPDSLAVSLGERATINCRASKSVSAAGYNFMHWYQQKPGQPPKLLIYYASNLESGVPDRFSGSGSGTDFTLTISSLQAEDVVVYYCLHSREFPFTFGGGTKVEIK

SEQ ID NO: 30 - DNA, 333 bp, Artificial
  Humanised VL L3 nucleotide sequence - leto codon optimised
gacatcgtgatgactcagagccccgatagcctggccgtgagcctgggcgaaagggccaccatcaactgcagggccagcaagagcgtgagcgctgccggctacaacttcatgcactggtaccagcagaagcccggccagccccccaagctgctgatctactacgcctccaacctggagagcggcgtgccagacaggttcagcggatctggcagcggcaccgacttcaccctgaccatctcaagcctgcaggccgaggacgtcgccgtgtactactgcctgcacagcagggagttccccttcacctttggcggcggcaccaacgtggagatcaag

SEQ ID NO: 31 - protein, 111 aa, Artificial
  Humanised VL L3 amino acid sequence
DIVMTQSPDSLAVSLGERATINCRASKSVSAAGYNFMHWYQQKPGQPPKLLIYYASNLESGVPDRFSGSGSGTDFTLTISSLQAEDVAVYYCLHSREFPFTFGGGTNVEIK

SEQ ID NO: 32 - DNA, 333 bp, Artificial
  Humanised VL L4 nucleotide sequence -leto codon optimised
gacatcgtgatgactcagagccccgatagcctggccgtgagcctgggcgaaagggccaccatcaactgcagggccagcaagagcgtgagcgctgccggctacaacttcatgcactggtaccagcagaagcccggccagccccccaaggtgctgatctactacgcctccaacctggagagcggcgtgccagacaggttcagcggatctggcagcggcaccgacttcaccctgaccatctcaagcctgcaggccgaggacgtcgtggtgtactactgcctgcacagcagggagttccccttcacctttggcggcggcaccaacgtggagatcaag

SEQ ID NO: 33 - protein, 111 aa, Artificial
  Humanised VL  L4 amino acid sequence
DIVMTQSPDSLAVSLGERATINCRASKSVSAAGYNFMHWYQQKPGQPPKVLIYYASNLESGVPDRFSGSGSGTDFTLTISSLQAEDVVVYYCLHSREFPFTFGGGTNVEIK

SEQ ID NO: 34 - DNA, 1353 bp, Artificial
  Mature H0 heavy chain nucleotide sequence -leto codon optimised
gaggtgcagctggtggaaagcggcggcggcctggtccagcccggcgggagcctgagactctcttgcgccgctagcggcttcaccttcagcaactacgccatgagctgggtgaggcaggcccccggcaagggcctggagtgggtggccaccatcagcgacggcggcagcttcacctactatctggacaacgtgaggggcaggttcaccatcagcagggacaacgccaagaacagcctgtacctgcagatgaacagcctgagggccgaggataccgccgtgtactactgcgccagggacgtcggccacaccaccttctggtacttcgacgtctggggcaggggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 35 - protein, 451 aa, Artificial
  Mature H0 heavy chain amino acid sequence
EVQLVESGGGLVQPGGSLRLSCAASGFTFSNYAMSWVRQAPGKGLEWVATISDGGSFTYYLDNVRGRFTISRDNAKNSLYLQMNSLRAEDTAVYYCARDVGHTTFWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 654 bp, Artificial
  Mature L1 light chain nucleotide sequence - leto codon optimised
gacatcgtgatgactcagagccccgatagcctggccgtgagcctgggcgaaagggccaccatcaactgcagggccagcaagagcgtgagcgctgccggctacaacttcatgcactggtaccagcagaagcccggccagccccccaaggtgctgatctactacgcctccaacctggagagcggcgtgccagacaggttcagcggatctggcagcggcaccgacttcaccctgaccatctcaagcctgcaggccgaggacgtcgccgtgtactactgcctgcacagcagggagttccccttcacctttggcggcggcaccaaggtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 37 - protein, 218 aa, Artificial
  Mature L1 light chain amino acid sequence
DIVMTQSPDSLAVSLGERATINCRASKSVSAAGYNFMHWYQQKPGQPPKVLIYYASNLESGVPDRFSGSGSGTDFTLTISSLQAEDVAVYYCLHSREFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 363 bp, Artificial
  Humanised VH variant H0 (IGHV3_23 CDRH1) nucleotide sequence-leto codon optimised
gaggtgcagctggtggaaagcggcggcggcctggtccagcccggcgggagcctgagactctcttgcgccgctagcggcttcaccttcagcagctacgccatgagctgggtgaggcaggcccccggcaagggcctggagtgggtggccaccatcagcgacggcggcagcttcacctactatctggacaacgtgaggggcaggttcaccatcagcagggacaacgccaagaacagcctgtacctgcagatgaacagcctgagggccgaggataccgccgtgtactactgcgccagggacgtcggccacaccaccttctggtacttcgacgtctggggcaggggcacactagtgaccgtgtccagc

SEQ ID NO: 39 - protein, 121 aa, Artificial
  Humanised VH variant H0 (IGHV3_23 CDRH1) amino acid sequence
EVQLVESGGGLVQPGGSLRLSCAASGFTFSSYAMSWVRQAPGKGLEWVATISDGGSFTYYLDNVRGRFTISRDNAKNSLYLQMNSLRAEDTAVYYCARDVGHTTFWYFDVWGRGTLVTVSS

SEQ ID NO: 40 - DNA, 1353 bp, Artificial
  Mature H0 (IGHV3_23 CDRH1) heavy chain nucleotide sequence - leto codon optimised
gaggtgcagctggtggaaagcggcggcggcctggtccagcccggcgggagcctgagactctcttgcgccgctagcggcttcaccttcagcagctacgccatgagctgggtgaggcaggcccccggcaagggcctggagtgggtggccaccatcagcgacggcggcagcttcacctactatctggacaacgtgaggggcaggttcaccatcagcagggacaacgccaagaacagcctgtacctgcagatgaacagcctgagggccgaggataccgccgtgtactactgcgccagggacgtcggccacaccaccttctggtacttcgacgtctggggcaggggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 41 - protein, 451 aa, Artificial
  Mature H0 (IGHV3_23 CDRH1) heavy chain amino acid sequence
EVQLVESGGGLVQPGGSLRLSCAASGFTFSSYAMSWVRQAPGKGLEWVATISDGGSFTYYLDNVRGRFTISRDNAKNSLYLQMNSLRAEDTAVYYCARDVGHTTFWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 5 aa, Human
SYAMS

SEQ ID NO: 43 - protein, 7 aa, Human
KASSLES

SEQ ID NO: 44 - DNA, 759 bp, Human
atgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtag

SEQ ID NO: 45 - protein, 252 aa, Human
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 46 - protein, 902 aa, Artificial
  mAb 2 Heavy Chain amino acid sequence
EVQLVQSGAEVKKPGASVKVSCKASGYIFTDYNMDWVRQAPGQKLEWIGDINPNNGGTIDNQKFKDRATLTVDKSTSTVYMELSSLRSEDTAVYYCARGIYYYGSHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEVQLVQSGAEVKKPGASVKVSCKASGYIFTDYNMDWVRQAPGQKLEWIGDINPNNGGTIDNQKFKDRATLTVDKSTSTVYMELSSLRSEDTAVYYCARGIYYYGSHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 213 aa, Artificial
  mAb 2 Light Chain amino acid sequence
EIVLTQSPSSLSASVGDRVTITCSATSSVSVMHWFQKKPGKAPKRWIYDTSKLASGVPSRFSGSGSGTDYTLTISSLQPEDFATYYCQQWSSNPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC